(19) United States
(12) Patent Application Publication
(10) Pub. No.: US 2018/0193276 A1
(43) Pub. Date: Jul. 12, 2018

(54) DOSAGE FORMS OF HALOFUGINONE AND METHODS OF USE

(71) Applicant: AKASHI THERAPEUTICS, INC., Cambridge, MA (US)

(72) Inventors: Ernest D. Bush, Rockaway, NJ (US); Diane Mcguire, Franklin, MA (US); Marc B. Blaustein, Newton, MA (US)

(21) Appl. No.: 15/709,725

(22) Filed: Sep. 20, 2017

Related U.S. Application Data
(63) Continuation of application No. 14/389,061, filed on Sep. 29, 2014, now abandoned.
(60) Provisional application No. 61/798,784, filed on Mar. 15, 2013.
(60) Provisional application No. 61/617,356, filed on Mar. 29, 2012.

(51) Int. Cl. A61K 9/48; A61K 9/28

(52) U.S. Cl. CPC A61K 9/4891; A61K 9/0095; A61K 9/2846

(57) ABSTRACT

Oral and parenteral dosage forms comprising halofuginone, including enteric-coated solid oral dosage forms, subcutaneous dosage forms and intravenous dosage forms, for administration to subjects in need thereof, e.g., subjects having been identified with musculoskeletal disorders, fibrotic diseases, malaria, or cancer are described herein.

### CROSS-REFERENCE TO RELATED APPLICATIONS

The present application claims priority to U.S. Provisional Application No. 61/617,356, filed Mar. 29, 2012 and U.S. Provisional Application No. 61/798,784, filed Mar. 15, 2013, the contents of each of which are hereby incorporated by reference in their entireties.

### BACKGROUND

Halofuginone is a halogenated derivative of febrifugine, an alkaloid isolated from the plant Dichroa febrifuga. Halofuginone includes two carbon stereocenters. In some embodiments one or more of the carbon stereocenters is enriched for one or more stereoisomer. In some embodiments, one or more stereocenters are present to provide a racemic mixture of halofuginone. Halofuginone is depicted structurally as a compound of formula (I):

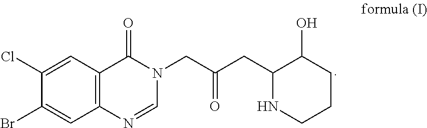 [SMILES: O=C(CC1NCCCC1O)Cn1cnc2cc(Br)c(Cl)cc2c1=O]

The compound of formula (I) includes two stereoisomers of halofuginone as shown below:

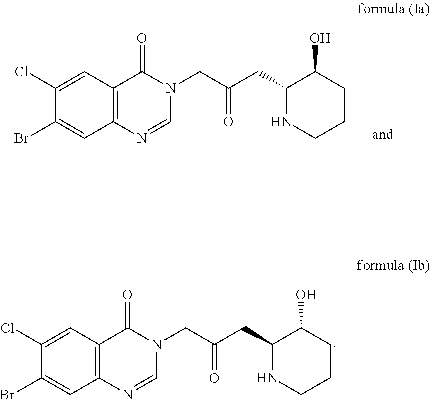 [SMILES: O=C(C[C@H]1NCCC[C@@H]1O)Cn1cnc2cc(Br)c(Cl)cc2c1=O ; O=C(C[C@@H]1NCCC[C@H]1O)Cn1cnc2cc(Br)c(Cl)cc2c1=O]

Halofuginone also includes two cis isomers (not depicted here). In some embodiments, the halofuginone mixture predominately includes one or more trans isomers as shown in formula (Ia) or (Ib) above (e.g greater than 50%, greater than 60%, greater than 70%, greater than 80%) of the compounds in the halofuginone have a trans configuration as shown in formula (Ia) or (Ib). Halofuginone has been used in the treatment of coccidiosis (a parasitic disease of the intestinal tract) in domesticated animals. More recently, studies have indicated that halofuginone may also be useful in treating a variety of fibrotic diseases, e.g., muscular dystrophy and scleroderma, vascular diseases, e.g., restenosis, cancer, e.g., metastatic cancer, e.g., metastatic breast cancer, and autoimmune diseases, e.g., multiple sclerosis.

### SUMMARY

Described herein are forms of halofuginone and pharmaceutically acceptable salts thereof, which can be used to treat a disorder described herein. In embodiments, the dosage form or route of administration can reduce or limit stomach exposure to halofuginone (e.g., halofuginone in a form that can cause irritation to the stomach). Exemplary forms of halofuginone are oral dosage forms comprising halofuginone, e.g., solid oral dosage forms comprising halofuginone, enteric-coated solid oral dosage forms comprising halofuginone. In some embodiments, an oral dosage form described herein does not release halofuginone in the stomach of a subject upon administration. Also described herein are non-irritating formulations, including those that are not dosed through the gastrointestinal track, and thus avoid exposing the stomach to halofuginone. Exemplary non-irritating formulations include parenteral administrations such as a subcutaneous or an intravenous formulation. The dosage forms (e.g., oral dosage forms or parenteral dosage forms) can be used, for example, in the treatment of subjects having been identified with disorders such as muscular dystrophy, cancer, or malaria. Methods of treating a subject using these oral dosage forms e.g., treating a subject identified as having muscular dystrophy, cancer, or malaria, are also described. Despite known toxicities associated with oral administration of halofuginone, as well as the known instability of halofuginone in basic environments, the disclosed dosage forms (e.g., oral dosage forms) result in increased peak plasma concentrations (Cₘₐₓ) of halofuginone while reducing or eliminating the side effects observed in earlier clinical studies, e.g., nausea and vomiting.

In one aspect, an oral dosage form comprising halofuginone, or a pharmaceutically acceptable salt thereof, e.g., halofuginone hydrobromide, dissolvable in a basic solution (e.g., pH greater than 6.8, greater than 8, greater than 8.5), wherein not more than 10% of the oral dosage form dissolves in an acidic solution (2 hours in 0.1 N hydrochloric acid) (e.g., pH less than 7, less than 6, less than 5, less than 4, less than 3). In one embodiment, the oral dosage form comprises 0.01 to 10 mg of halofuginone, or a pharmaceutically acceptable salt thereof, e.g., halofuginone hydrobromide. In one embodiment, the oral dosage form comprises 0.05 to 2 mg of halofuginone, or a pharmaceutically acceptable salt thereof, e.g., halofuginone hydrobromide. In one embodiment, the pharmaceutically acceptable salt of halofuginone is halofuginone hydrobromide. In one embodiment, the oral dosage form is an enteric-coated solid oral dosage form. In one embodiment, the enteric coating comprises poly(methacrylic acid-co-ethyl acrylate).

In another aspect, the disclosure provides for a method of treating a disorder in a patient in need thereof, the method comprising administering an enteric-coated oral dosage form comprising 0.01 to 10 mg of halofuginone, or a pharmaceutically acceptable salt thereof, e.g., halofuginone hydrobromide, to thereby treat the disorder. In one embodiment, the oral dosage form comprises 0.05 to 2 mg of halofuginone or a pharmaceutically acceptable salt thereof, e.g., halofuginone hydrobromide. In one embodiment, the pharmaceutically acceptable salt of halofuginone is halofuginone hydrobromide. In one embodiment, the oral dosage form is an enteric-coated solid oral dosage form. In one embodiment, the enteric coating comprises poly(methacrylic acid-co-ethyl acrylate). In one embodiment, the disorder is a musculoskeletal disorder. In one embodiment, the musculoskeletal disorder is muscular dystrophy. In one embodiment, the muscular dystrophy is selected from the group consisting of Duchenne MD, Becker MD, Emery-Dreifuss MD, Limb-Girdle MD, facioscapulohumeral MD, myotonic dystrophy, oculopharyngeal MD, distal MD, and congenital MD. In one embodiment, the muscular dystrophy is Duchenne muscular dystrophy. In one embodiment, the disorder is cancer. In one embodiment, the disorder is metastatic cancer.

In another aspect, the disclosure provides for an oral dosage form comprising halofuginone, wherein when administered to a subject, results in a maximum concentration (Cₘₐₓ) of at least 3 ng halofuginone/ml of plasma in the subject, when the oral dosage form is administered to the subject at a dose of 0.1 mg halofuginone per kg of subject weight. In one embodiment, the subject is a mammal. In one embodiment, the subject is a human. In one embodiment, the subject has been identified as having a disorder that would benefit from the administration of halofuginone. In one embodiment, the subject has been identified with a disorder selected from a fibrotic disease, an autoimmune disease, a vascular disease, malaria, and cancer.

In another aspect, the disclosure provides for an oral dosage form comprising halofuginone, wherein when administered to a subject, results in a maximum concentration (Cₘₐₓ) of at least 6 ng halofuginone/ml of plasma in the subject, when the oral dosage form is administered to the subject at a dose of 0.2 mg halofuginone per kg of subject weight. In one embodiment, the subject is a mammal. In one embodiment, the subject is a human. In one embodiment, the subject has been identified as having a disorder which would benefit from the administration of halofuginone. In one embodiment, the subject has been identified with a disorder selected from a fibrotic disease, an autoimmune disease, a vascular disease, and cancer.

In another aspect, the disclosure provides for an oral dosage form comprising halofuginone, or a pharmaceutically acceptable salt thereof, e.g., halofuginone hydrobromide, for administration to a subject at a dose of at least 0.05 mg halofuginone per kilogram of subject weight, wherein the subject does not experience gastrointestinal distress (e.g., nausea, vomiting, pain) within eight hours (e.g., within six hours, e.g., within four hours, e.g., within two hours, e.g., within one hour) of administration. In one embodiment, the dosage form is administered to a subject at a dose of at least 0.1 mg/kg. In one embodiment, the subject is a mammal. In one embodiment, the subject is a human. In one embodiment, the subject has been identified as having a disorder that would benefit from the administration of halofuginone. In one embodiment, the subject has been identified with a disorder selected from a fibrotic disease, an autoimmune disease, a vascular disease, malaria, and cancer.

In another aspect, the disclosure provides for a method of administering an effective amount of halofuginone, or a pharmaceutically acceptable salt thereof, e.g., halofuginone hydrobromide, to a subject in need thereof, the method comprising administering an enteric-coated solid oral dosage form comprising 0.01 to 10 mg of halofuginone, or a pharmaceutically acceptable salt thereof, e.g., halofuginone hydrobromide, wherein the subject does not experience gastrointestinal distress (e.g., nausea, vomiting, pain) within eight hours (e.g., within six hours, e.g., within four hours, e.g., within two hours, e.g., within one hour) of administration. In one embodiment, the dosage form is administered to a subject at a dose of at least 0.1 mg/kg. In one embodiment, the subject is a mammal. In one embodiment, the subject is a human. In one embodiment, the subject has been identified as having a disorder which would benefit from the administration of halofuginone. In one embodiment, the subject has been identified with a disorder selected from a fibrotic disease, an autoimmune disease, a vascular disease, malaria, and cancer.

In another aspect, the disclosure provides for an oral dosage form comprising halofuginone, or a pharmaceutically acceptable salt thereof, e.g., halofuginone hydrobromide, wherein when administered to a subject, results in an area under the plasma concentration time curve (AUC) of at least 40 ng·hour/mL, at a dose of 0.2 mg halofuginone/kg of subject weight. In some embodiments, the subject is a mammal. In some embodiments, the mammal is a human. In some embodiments, the oral dosage form comprises an enteric coating.

In one aspect, the disclosure provides for a parenteral dosage form of halofuginone or a pharmaceutically acceptable salt thereof, such as an exemplary parenteral dosage form described herein. In some embodiments, the halofuginone is formulated for subcutaneous administration. In some embodiments, when formulated for a subcutaneous administration, the halofuginone or a pharmaceutically acceptable salt thereof, is formulated into an aqueous solution. In some embodiments, the halofuginone is formulated for intravenous administration. In some embodiments, when formulated for a intravenous administration, the halofuginone or a pharmaceutically acceptable salt thereof, is formulated into an aqueous solution. In some embodiments, the dosage form is used to treat a subject that has been identified as having a disorder selected from a fibrotic disease, an autoimmune disease, a vascular disease, malaria, and cancer.

In methods where the halofuginone or a pharmaceutically acceptable salt thereof is formulated for parenteral administration, the halofuginone or a pharmaceutically acceptable salt thereof is formulated as a solution (e.g., an aqueous solution). In some embodiments, the concentration of the halofuginone or a pharmaceutically acceptable salt thereof in the solution is from about 0.05 mg/mL to about 1 mg/ml (e.g., from about 0.1 mg/mL to about 0.8 mg/mL, e.g., about 0.2 mg/mL, about 0.3 mg/mL, about 0.4 mg/mL, about 0.5 mg/mL, about 0.6 mg/mL, about 0.7 mg/mL, or about 0.8 mg/mL). In some embodiments, the formulation is used to treat a subject that has been identified as having a disorder selected from a fibrotic disease, an autoimmune disease, a vascular disease, malaria, and cancer.

In some embodiments, when the halofuginone or a pharmaceutically acceptable salt thereof is dosed parenterally, the halofuginone or a pharmaceutically acceptable salt thereof is administered in an amount from about 0.01 mg/kg to about 0.5 mg/kg, e.g., from about 0.01 mg/kg to about 0.1 mg/kg. For example, when the halofuginone or a pharmaceutically acceptable salt thereof is administered subcutaneously, it can be administered at a dose from about 0.01 mg/kg to about 0.05 mg/kg (e.g., about 0.03 mg/kg). In some embodiments, when the halofuginone or a pharmaceutically acceptable salt thereof is administered intravenously, the halofuginone or a pharmaceutically acceptable salt thereof can be administered at a dose from about 0.01 to about 0.1 mg/kg (e.g., about 0.05 mg/kg). In some embodiments, the dosage form is used to treat a subject that has been identified as having a disorder selected from a fibrotic disease, an autoimmune disease, a vascular disease, malaria, and cancer.

### BRIEF DESCRIPTION OF THE DRAWINGS

FIGS. 1A and 1B show the % dissolution of enteric-coated and non-enteric coated (uncoated) capsules containing halofuginone in simulated gastric and intestinal fluids, respectively.

FIG. 2 shows concentrations of halofuginone in dog plasma as a function of time after administration of an aqueous solution at a dose of 0.15 mg/kg.

FIG. 3 shows concentrations of halofuginone in dog plasma as a function of time after administration of a non-enteric-coated capsule at a dose of 0.1 mg/kg.

FIG. 4 shows concentrations of halofuginone in dog plasma as a function of time after administration of an enteric-coated capsule at a dose of 0.1 mg/kg.

FIG. 5 shows concentrations of halofuginone in dog plasma as a function of time after administration of a non-enteric-coated capsule at a dose of 0.2 mg/kg.

FIG. 6 shows concentrations of halofuginone in dog plasma as a function of time after administration of an enteric-coated capsule at a dose of 0.2 mg/kg.

FIG. 7 shows the Study Design of dog studies administered with oral and parenteral forms of halofuginone.

FIG. 8 shows a line graph depicting the average concentrations (±SD) of Halofuginone hydrobromide following administration of an oral tablet (˜0.15 mg/kg), oral solution (0.15 mg/kg), subcutaneous solution (0.03 mg/kg), or intravenous solution (0.05 mg/kg).

### DETAILED DESCRIPTION

This disclosure is not limited in its application to the details of the dosage forms or the specific order of preparation or administration of the dosage forms. The dosage forms described herein may be suitably prepared using other techniques and/or administered in various ways. Also, the phraseology and terminology used herein is for the purpose of description and should not be regarded as limiting.

### Definitions

The term “pharmaceutically acceptable carrier or adjuvant,” as used herein, refers to a carrier or adjuvant that may be administered to a subject, together with a compound of this invention, and which does not destroy the pharmacological activity thereof and is nontoxic when administered in doses sufficient to deliver a therapeutic amount of the compound.

The term, “pharmaceutically acceptable salts,” as used herein, refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the disclosure include the conventional non-toxic salts of the parent compound, e.g., halofuginone, formed, for example, from non-toxic inorganic or organic acids. The pharmaceutically acceptable salts of the disclosure can be synthesized from the parent compound, e.g., halofuginone, which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase, “pharmaceutically acceptable derivative or prodrug,” as used herein refers to any pharmaceutically acceptable salt, ester, salt of an ester, or other derivative of a compound, e.g., a hydrobromide salt, e.g., halofuginone hydrobromide, or halofuginone lactate, which, upon administration to a recipient, is capable of providing (directly or indirectly) a therapeutic agent. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

The term, “oral dosage form,” as used herein, refers to a composition or medium used to administer an agent, e.g., halofuginone, to a subject. Typically, an oral dosage form is administered via the mouth, however, “oral dosage form” is intended to cover any substance which is administered to a subject and is absorbed across a membrane, e.g., a mucosal membrane, of the gastrointestinal tract, including, e.g., the mouth, esophagus, stomach, small intestine, large intestine, and colon. For example, “oral dosage form” covers a solution which is administered through a feeding tube into the stomach. “Oral dosage forms” may be administered buccally or sublingually.

The term, “parenteral dosage form,” as used herein, refers to a composition or medium used to administer an agent, e.g., halofuginone, to a subject by way other than mouth or the gastrointestinal tract. Exemplary parenteral dosage forms or modes of administration include intranasally, buccally, intravenous, intramuscular, subcutaneous, intraparenteral, bucosal, sublingual, intraoccular, and topical (e.g., intravenous or subcutaneous).

The term, “treat” or “treatment,” as used herein, refers to the application or administration of a compound, alone or in combination with, a second compound to a subject, e.g., a subject, or application or administration of the compound to an isolated tissue or cell, e.g., cell line, from a subject, e.g., a subject, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to minimize at least one symptom of the disorder or to delay onset of at least one symptom of the disorder).

The phrase, “an amount of a compound effective to treat a disorder,” or a “therapeutically effective amount,” as used herein, refers to an amount of the compound which is effective, upon single or multiple dose administration to a subject, in treating a cell, or in curing, alleviating, relieving or improving a subject with a disorder beyond that expected in the absence of such treatment.

The term, “dissolvable,” as used here, refers to a compound or composition whereby at least 50% (wt/wt), e.g., 70%, e.g., 80%, e.g., 90%, e.g., 98% of the compound or composition goes into solution within 120 minutes when the compound or composition is placed in a preponderance of solvent, i.e., the compound or composition is placed in solvent at a ratio of at least 10:1 solvent:compound or composition (wt/wt).

As used herein, the term “subject” is intended to include human and non-human animals. Exemplary human subjects include a human subject having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, dog, cat, cow, pig, etc.

### Dosage Forms and Dose Protocols

Oral Dosage Forms and Dose Protocols

Described herein are oral dosage forms comprising halofuginone, or pharmaceutically acceptable salts thereof, e.g., halofuginone hydrobromide, e.g., solid oral dosage forms comprising halofuginone, e.g., enteric-coated solid oral dosage forms comprising halofuginone. The oral dosage forms can be used, for example, in the treatment of subjects having been identified with disorders such as muscular dystrophy, malaria, or cancer. Methods of treating a subject using these oral dosage forms e.g., treating a subject identified as having muscular dystrophy or cancer, are also described. Despite known toxicities associated with oral administration of halofuginone, as well as the known instability of halofuginone in basic environments, the disclosed oral dosage forms result in increased peak plasma concentrations (Cₘₐₓ) of halofuginone while reducing or eliminating the side effects observed in earlier clinical studies, e.g., nausea and vomiting.

In another aspect, the oral dosage form comprises halofuginone, or a pharmaceutically acceptable salt thereof, e.g., halofuginone hydrobromide, dissolvable in a basic solution (e.g., pH greater than 6.8), wherein not more than 10% of the oral dosage form dissolves in an acidic solution (e.g., pH less than 6.8). In one embodiment, the oral dosage form comprises 0.01 to 10 mg of halofuginone or a pharmaceutically acceptable salt thereof, e.g., halofuginone hydrobromide. In one embodiment, the oral dosage form comprises 0.1 to 1 mg of halofuginone, or a pharmaceutically acceptable salt thereof, e.g., halofuginone hydrobromide. In one embodiment, the pharmaceutically acceptable salt of halofuginone is halofuginone hydrobromide. In one embodiment, the oral dosage form is an enteric-coated solid oral dosage form. In one embodiment, the enteric coating comprises poly(methacrylic acid-co-ethyl acrylate).

In another aspect, the disclosure provides for a method of treating a disorder in a patient in need thereof, the method comprising administering an enteric-coated oral dosage form comprising 0.01 to 10 mg of halofuginone, or a pharmaceutically acceptable salt thereof, e.g., halofuginone hydrobromide, to thereby treat the disorder. In one embodiment, the oral dosage form comprises 0.05 to 1 mg of halofuginone, or a pharmaceutically acceptable salt thereof, e.g., halofuginone hydrobromide. In one embodiment, the pharmaceutically acceptable salt of halofuginone is halofuginone hydrobromide. In one embodiment, the oral dosage form is an enteric-coated solid oral dosage form. In one embodiment, the enteric coating comprises poly(methacrylic acid-co-ethyl acrylate). In one embodiment, the disorder is a musculoskeletal disorder. In one embodiment, the musculoskeletal disorder is muscular dystrophy. In one embodiment, the muscular dystrophy is selected from the group consisting of Duchenne MD, Becker MD, Emery-Dreifuss MD, Limb-Girdle MD, facioscapulohumeral MD, myotonic dystrophy, oculopharyngeal MD, distal MD, and congenital MD. In one embodiment, the muscular dystrophy is Duchenne muscular dystrophy. In one embodiment, the disorder is cancer. In one embodiment, the disorder is metastatic cancer. In one embodiment, the disorder is a parasitic disorder, e.g., malaria. In one embodiment, the disorder is a fibrotic disease, e.g., scleroderma. In one embodiment the disorder is an autoimmune disease, e.g., multiple sclerosis.

In another aspect, described herein is an oral dosage form comprising halofuginone, or a pharmaceutically acceptable salt thereof, e.g., halofuginone hydrobromide, wherein when administered to a subject, results in a maximum concentration (Cₘₐₓ) of at least 3 ng halofuginone/mL of plasma in a subject, when the oral dosage form is administered to the subject at a dose of 0.075 mg halofuginone per kg of subject weight. In one embodiment, the subject is a mammal. In one embodiment, the subject is a human. In one embodiment, the subject has been identified as having a disorder which would benefit from the administration of halofuginone. In one embodiment, the subject has been identified with a disorder selected from a fibrotic disease, an autoimmune disease, a vascular disease, a parasitic disease, e.g., malaria, and cancer.

In another aspect, an oral dosage form comprising halofuginone which results in a maximum concentration (Cₘₐₓ) of at least 6 ng halofuginone/ml of plasma in a subject, when the oral dosage form is administered to the subject at a dose of 0.2 mg halofuginone per kg of subject weight. In one embodiment, the subject is a mammal. In one embodiment, the subject is a human. In one embodiment, the subject has been identified as having a disorder which would benefit from the administration of halofuginone. In one embodiment, the subject has been identified with a disorder selected from a fibrotic disease, an autoimmune disease, a vascular disease, a parasitic disease, e.g., malaria, and cancer.

In another aspect, the disclosure provides for an oral dosage form comprising halofuginone, or a pharmaceutically acceptable salt thereof, e.g., halofuginone hydrobromide, for administration to a subject at a dose of at least 0.05 mg halofuginone per kg of subject weight, wherein the subject does not experience gastrointestinal distress (e.g., nausea, vomiting, pain) within eight hours (e.g., within six hours, e.g., within four hours, e.g., within two hours, within 1 hour) of administration. In one embodiment, the dosage form is administered to a subject at a dose of at least 0.1 mg/kg. In one embodiment, the subject is a mammal. In one embodiment, the subject is a human. In one embodiment, the subject has been identified as having a disorder which would benefit from the administration of halofuginone. In one embodiment, the subject has been identified with a disorder selected from a fibrotic disease, an autoimmune disease, a vascular disease, a parasitic disease, e.g., malaria, and cancer.

An oral dosage form, e.g., an oral dosage form described herein, may be administered one, two, three, four, five, six, seven, eight, or more times daily. A dosage regimen may continue for one week, two weeks, one month, or longer. In some embodiments, the oral dosage form is administered at least once daily for two weeks. In some embodiments, the oral dosage form is administered twice daily for two weeks or longer.

The oral dosage forms described herein, e.g., enteric-coated solid dosage forms, can be administered in combination with one or more actives or other therapies. The oral dosage forms may be administered at the same time or sequentially with one or more actives or other therapies. The oral dosage forms may be administered in a staggered fashion, e.g., each drug is taken twice daily, with six hours between administration of each different drug. In some embodiments, oral dosage forms described herein may be co-administered with anti-emetic drugs, e.g., Mirtazapine. Oral dosage forms described herein may be administered with additional therapeutic compounds, such as anti-inflammatory agents, anti-viral agents, anti-cancer agents, or anti-fibrotic agents.

Parenteral Dosage Forms and Dose Protocols

In one aspect, the disclosure provides for a parenteral dosage form of halofuginone or a pharmaceutically acceptable salt thereof, such as an exemplary parenteral dosage form described herein. In some embodiments, the halofuginone is formulated for subcutaneous administration. In some embodiments, when formulated for a subcutaneous administration, the halofuginone or a pharmaceutically acceptable salt thereof, is formulated into an aqueous solution. In some embodiments, the halofuginone is formulated for intravenous administration. In some embodiments, when formulated for a intravenous administration, the halofuginone or a pharmaceutically acceptable salt thereof, is formulated into an aqueous solution.

In methods where the halofuginone or a pharmaceutically acceptable salt thereof is formulated for parenteral administration, the halofuginone or a pharmaceutically acceptable salt thereof is formulated as a solution (e.g., an aqueous solution). In some embodiments, the concentration of the halofuginone or a pharmaceutically acceptable salt thereof is the solution is from about 0.05 mg/mL to about 1 mg/ml (e.g., from about 0.1 mg/mL to about 0.8 mg/mL, e.g., about 0.2 mg/mL, about 0.3 mg/mL, about 0.4 mg/mL, about 0.5 mg/mL, about 0.6 mg/mL, about 0.7 mg/mL, or about 0.8 mg/mL).

In some embodiments, when the halofuginone or a pharmaceutically acceptable salt thereof is dosed parenterally, the halofuginone or a pharmaceutically acceptable salt thereof is administered in an amount from about 0.01 mg/kg to about 0.5 mg/kg, e.g., from about 0.01 mg/kg to about 0.1 mg/kg. For example, when the halofuginone or a pharmaceutically acceptable salt thereof is administered subcutaneously, it can be administered at a dose from about 0.01 mg/kg to about 0.05 mg/kg (e.g., about 0.03 mg/kg). In some embodiments, when the halofuginone or a pharmaceutically acceptable salt thereof is administered intravenously, the halofuginone or a pharmaceutically acceptable salt thereof can be administered at a dose from about 0.01 to about 0.1 mg/kg (e.g., about 0.05 mg/kg). In one embodiment, the subject is being treated for a musculoskeletal disorder. In one embodiment, the musculoskeletal disorder is muscular dystrophy. In one embodiment, the muscular dystrophy is selected from the group consisting of Duchenne MD, Becker MD, Emery-Dreifuss MD, Limb-Girdle MD, facioscapulohumeral MD, myotonic dystrophy, oculopharyngeal MD, distal MD, and congenital MD. In one embodiment, the muscular dystrophy is Duchenne muscular dystrophy. In one embodiment, the subject is being treated for a cancer. In one embodiment, the disorder is metastatic cancer. In one embodiment, the disorder is a parasitic disorder, e.g., malaria. In one embodiment, the disorder is a fibrotic disease, e.g., scleroderma. In one embodiment the disorder is an autoimmune disease, e.g., multiple sclerosis.

### Halofuginone

Quinazolinone derivatives have been used for some time to treat a variety of disorders, e.g., intestinal parasitic diseases, e.g., coccidiosis. Halofuginone, (7-bromo-6-chloro-3-[3-(3-hydroxy-2-piperidinyl)-2-oxopropyl]-4(3H)-quinazolinone), an analog of a plant alkaloid originally isolated from the plant Dichroa febrifuga, is the quinazolinone derivative most widely used as a coccidiostat. U.S. Pat. Nos. 4,824,847; 4,855,299; 4,861,758 and 5,215,993, incorporated by reference herein in their entireties, all relate to the coccidiocidal properties of halofuginone.

In addition to coccidiocidal properties, it was more recently discovered that quinazolinone derivatives, e.g., halofuginone, can inhibit collagen synthesis, and are useful for the treatment of conditions such as scleroderma and graft-versus-host disease (GVHD). See, e.g., U.S. Pat. No. 5,449,678, incorporated by reference herein in its entirety. Furthermore, U.S. Pat. No. 5,891,879, incorporated by reference herein in its entirety, discloses that quinazolinone derivatives, e.g., halofuginone, are effective in treating restenosis and other forms of vascular disease.

Halofuginone-containing pharmaceutical compositions, including oral dosage forms, have been administered in attempts to treat these disorders, as well as malaria. See Jiang et al., “Antimalarial Activities and Therapeutic Properties of Febrifugine Analogs,” Antimicrobial Agents and Chemotherapy, March 2005, p. 1169-1176. However, halofuginone has not been commercially-successful because it is not tolerated in the gastrointestinal tract. For example, Jiang et al. report widespread gastrointestinal tract injuries, e.g., intestinal hemorrhage, in mice that were administered halofuginone at a dose of 5 mg/kg. In contrast, mice administered halofuginone subcutaneously did not experience the same levels of gastrointestinal tract injuries, however, because halofuginone is much more toxic when administered subcutaneously, the allowable dosages were much smaller, and were not effective.

Additional disclosures related to administration of halofuginone are disclosed in U.S. Patent Publication Nos. 20070184082 and 20050208134 both of which are incorporated herein by reference in their entireties.

### Oral Formulations

Oral dosage forms may comprise, in addition to halofuginone, a pharmaceutically acceptable carrier, and may optionally further comprise one or more pharmaceutically acceptable excipients, such as, for example, binding agents, stabilizers, diluents, surfactants, flavors, and odorants.

Pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the oral dosage form is a liquid. Saline solutions and aqueous dextrose and glycerol solutions can also be employed as liquid carriers. Oral dosage forms may be manufactured by processes well known in the art, e.g., by means of conventional mixing, dissolving, granulating, surface deposition, dragee-making, levigating, emulsifying, encapsulating, entrapping or lyophilizing processes. Further techniques for formulation and administration of active ingredients may be found in “Remington's Pharmaceutical Sciences,” Mack Publishing Co., Easton, Pa., latest edition, which is incorporated herein by reference as if fully set forth herein. Oral dosage forms for use in accordance with the present invention thus may be formulated in conventional manner using one or more pharmaceutically acceptable carriers comprising excipients and auxiliaries, which facilitate processing of the active ingredients into preparations which, can be used pharmaceutically.

For oral administration, the active ingredients, e.g., halofuginone, e.g., halofuginone hydrobromide, can be formulated readily by combining the active ingredients with pharmaceutically acceptable carriers well known in the art. Such carriers enable the active ingredients of the invention to be formulated as tablets, pills, dragees, capsules, liquids, gels, syrups, slurries, powders or granules, suspensions or solutions in water or non-aqueous media, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients such as thickeners, diluents, flavorings, dispersing aids, emulsifiers, binders or preservatives may be desirable.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1). Lower or higher doses than those recited above may be required. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

Oral dosage forms may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

### Enteric Coatings

Enteric coatings have been used for many years to delay the release of the drug from the dosage forms. Depending on the composition and/or thickness, the enteric coatings are resistant to acidic gastric fluids but are soluble at higher pH in the intestine. Therefore, enteric coated oral dosage forms do not release the drug in the acidic gastric fluids where the drug is susceptible to degradation. The enteric coating polymer may be selected from polymers soluble at pH existing in the upper part of the small intestine or in the latter part of the small intestine and accordingly the release of the drug is delayed by a time period required for the dosage form to transit to these parts of the intestine. A variety of enteric coatings are known, including, but not limited to waxes, shellacs, polymers, and plant fibers. Specific enteric coatings include methyl acrylate-methacrylic acid copolymers, cellulose acetate succinate, hydroxy propyl methyl cellulose phthalate, hydroxy propyl methyl cellulose acetate succinate, polyvinyl acetate phthalate, methyl methacrylate-methacrylic acid copolymers, sodium alginate, and stearic acid. An enteric coating may be applied to a solid oral dosage form, e.g., a capsule or tablet, using a variety of known techniques, e.g., spray coating or pan coating. In some embodiments, the enteric coating can be: methylcellulose; ethylcellulose hydroxyethylcellulose; hydroxypropylmethylcellulose (HPMC); sodium carboxymethylcellulose; agar-agar; carob gum; alginates; molasses; polysaccharides of mannose and galactose; chitosan; modified starches; aliphatic poly (esters); poly anhydrides; polyhydroxyethyle methylacrylate (PHEMA); cross-linked polyvinyl alcohol (PVA); cross-linked polyvinyl pyrrolidone (PVP); polyethylene oxide (PEO); polyacrylamide (PA); polyethylene glycol (PEG); polyvinyl alcohol (PVA); polyvinyl pyrrolidone (PVP); hydroxypropyl methyl cellulose (HPMC); polylactic acid (PLA); polyglycolic acid (PGA); polycaprolactone (PCL); polyanhydrides; polyorthoesters; polyethylene vinyl acetate (PVA); polydimethyl siloxane (PDS); polyether urethane (PEU); polyvinyl chloride (PVC); cellulose acetate (CA); ethyl cellulose (EC); polycarbophil; sodium carboxymethyl cellulose; polyacrylic acid; tragacanth; methyl cellulose; pectin; xanthan gum; guar gum; and Karaya gum.

### Parenteral Formulations

In some embodiments, halofuginone or a pharmaceutically acceptable salt thereof is formulated for parenteral administration containing a halofuginone or a pharmaceutically acceptable salt thereof, and a pharmaceutical excipient suitable for parenteral administration. Exemplary forms of parenteral administration include intravenous, intraarterial, subcutaneous, intramuscular, intravascular, intraperitoneal or infusion.

The forms in which the halofuginone or a pharmaceutically acceptable salt thereof can be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline can also be used for injection. Exemplary excipients include ethanol, glycerol, propylene glycol, liquid polyethylene glycol, cyclodextrin derivatives, and vegetable oils.

Sterile injectable solutions can be prepared by incorporating halofuginone or a pharmaceutically acceptable salt thereof in the required amount in the appropriate solvent with one or more excipients, followed by filtered sterilization. Dispersions can be prepared by incorporating a sterilized halofuginone or a pharmaceutically acceptable salt thereof into a sterile vehicle. An injectable formulation can be sterilized, for example, by filtration through a bacterial-retaining filter, or by incorporating sterilizing agents in the form of sterile solid compositions which can be dissolved or dispersed in sterile water or other sterile injectable medium prior to use. Injectable compositions can contain from about 0.1 to about 5% w/w of a compound as disclosed herein.

### Disorders

A formulation of halofuginone or a pharmaceutically acceptable salt thereof (e.g., an oral dosage form described herein or a parenteral dosage form described herein) can be used to treat a variety of disorders, e.g., disorders described herein. In general, subjects having been identified with musculoskeletal disorders, disorders relating to the formation of collagen, or disorders relating to or facilitated by the formation of blood vessels, e.g., metastatic cancer, or an autoimmune disease may receive benefit from the administration of oral dosage forms comprising halofuginone.

### Fibrotic Disorders

As disclosed in Pines et al. Gen. Pharmac. Vol. 30, No. 4, pp. 445-450, 1998, progressive fibroproliferative diseases such as liver cirrhosis, pulmonary and kidney fibrosis, scleroderma, etc., exhibit excessive production of connective tissues, results in destruction of normal tissue architecture and function. The fibrotic reaction is thought to involve the stimulative response of tissue cells resulting in increased proliferation as well as extracellular matrix (ECM) deposition. Collagen was found to be a major ECM molecule synthesized in the fibrotic lesion. In some cases, such as in pulmonary and kidney fibrosis, the fibroblasts are thought to play a pivotal role (Rodemann and Bamberg, 1995; Rodemann et al., 1996). In normal and injured liver, other resident cells, such as stellate cells, were found to be the cellular source of collagen and other ECM molecules (Friedman, 1993). It is generally recognized that most treatments for these diseases have little effect upon the inexorable pathological progression. Moreover, in many of these diseases a chronic inflammatory process may provide a continuing stimulus to fibrogenesis.

In addition to the fibrotic diseases with excess collagen deposition, normal wound healing involves the formation of scars and fibrous tissue that consists largely of collagen fibrils. Collagen molecules are an integral part of fibrous elements or supramolecular structures in extracellular spaces that function as major components of the various connective tissues. Although moderate degrees of fibrous tissue are beneficial in wound repair, fibrous material often accumulates in excessive amounts and impairs the normal function of the affected tissue. Such excessive accumulation of collagen becomes an important event in scarring of the skin after burns or traumatic injury (Zhang et al., 1995b), in hypertrophic scars and in keloids (Friedman et al., 1993). The formation of postoperational adhesions, which is the major cause of intestinal obstruction (Menzies, 1993; Weibel and Manjo, 1973), sterility in females (Monk et al., 1994), and difficulties during subsequent recurring operations (Yaffe et al., 1980), is another example of the results of excessive collagen deposition.

### Muscular Dystrophy

Muscle fibrosis is a phenomenon that frequently occurs in diseased or damaged muscle. It is characterized by the excessive growth of fibrous tissue, which usually results from the body's attempt to recover from injury. Fibrosis impairs muscle function and causes weakness. The amount of muscle function loss generally increases with the extent of fibrosis. Fibrosis is usually progressive and can contribute to the patient's inability to carry out ordinary tasks of independent living, such as grasping objects or walking. Fibrosis commonly occurs as a result of muscular dystrophy, as well as due to other afflictions, such as denervation atrophy, a degradation of muscle tissue caused by loss of neural contact to a muscle. For some types of muscular dystrophy, such as Duchenne, fibrosis can result in death as the muscles of the diaphragm are affected (the diaphragm is a skeletal muscle which is involuntary rather than voluntary).

Muscular dystrophies are a heterogeneous group of genetic disorders characterized by the progressive loss of muscle strength and integrity. Dystrophic muscle shows variation in muscle fiber size, infiltration of connective and fatty tissue, and centrally located nuclei. The membranes of the fibers are fragile and extensive damage occurs, leading to necrosis and muscle wasting. Victims of muscular dystrophies, particularly Becker muscular dystrophy (BMD) and Duchenne muscular dystrophy (DMD), frequently suffer from increasing skeletal muscle fibrosis as the disease progresses.

The most common form of muscle dystrophy is the X-linked recessive DMD, a severely penetrating allelic manifestation which affects 1 in 3500 live males at birth; about a third of cases occur as de novo mutations in the infant (Emery A E. (1991) Neuromusc. Disord. 1:19-29). Usually the disease is diagnosed at 4-5 years of age and by 8-10 years, deterioration of the patient's condition necessitates wheelchair use. By their early teens, further neurological and cardiological symptoms are apparent. Progression of muscle degeneration and worsening clinical symptoms, lead to death in the late teens or early twenties, typically as a result of cardio-pulmonary complications due to fibrosis of the diaphragm.

The leading causes of death in DMD victims, respiratory and heart failure, result from weakness in diaphragm and myocardium muscles that are most affected by fibrosis (Finsterer, (2003) Cardiology 99:1-19). Fibrosis is characterised by an increase in extra-cellular matrix (ECM) constituents especially collagen type I. Both in DMD and Congenital muscular dystrophy (CMD), an increase in type I and III collagens were observed in the skeletal muscle (Hantai et al. (1985) Connect Tissue Res. 13:273-81 and Dunace, et al. (1980) Nature 284:470-472) leading to fibrosis, which correlated with muscle destruction (Zhao, et al. (2003) J. Patho. 201:149-59). The cardiac involvement in DMD is characterized pathologically by degeneration and fibrosis of the myocardium, probably due to myofibroblast activity, centering around the posterolateral wall of the left ventricle.

BMD is a less severe condition than DMD, characterized by slowly progressive muscle weakness of the legs and pelvis, again due to fibrosis of the muscles (although for BMD the skeletal muscles are more greatly affected). The advance of fibrosis often causes ever greater loss of mobility and a reduced life expectancy. At some point, the patient may become too weak to walk and takes to a wheelchair.

Both BMD and DMD are associated with defects in the dystrophin gene, the gene responsible for the production of dystrophin protein, which is a vital part of the dystrophin-glycoprotein complex. DMD is characterized by the near absence of dystrophin protein in skeletal muscles, while BMD results from different mutations in the same gene, resulting in decreased or damaged dystrophin. The presence of some dystrophin protects the muscles of those with BMD from degenerating as badly or as quickly as those of DMD victims.

As described in U.S. Patent Application No. US2010/0144766, incorporated herein by reference in its entirety, halofuginone has been shown to be effective in improving muscle physiology and/or reducing the pressure on muscle regeneration in mdx mice, a known murine model for studying muscular dystrophy, e.g., Duchenne muscular dystrophy.

### Vascular Disease

The pathogenesis of atherosclerosis involves abnormal migration and proliferation of smooth muscle cells (SMCs) infiltrated with macrophages and embedded in extracellular matrix (ECM) of adhesive glycoproteins, proteoglycans and collagens V. Fuster, et al., “The Pathogenesis of Coronary Artery Disease and the Acute Coronary Syndromes,” New Eng. J. Med., Vol. 326, pp. 242-250 (1992); R. Ross, “The Pathogenesis of Atherosclerosis: A Perspective for the 1990's,” Nature, Vol. 362, pp. 801-809 (1993). Under physiological conditions, the majority of arterial SMCs remains in the Go phase and cell growth is controlled by a balance between endogenous proliferation-stimulating and proliferation-inhibiting factors. Following endothelial cell perturbation due to atherogenic risk factors (i.e., hypertension, hyperlipoproteinemia, diabetes mellitus), platelets and non-platelet-derived growth factors and cytokines are released and stimulate monocyte and SMC migration as well as SMC proliferation (V. Fuster, et al., ibid.; R. Ross, ibid.). Among these growth factors are platelet-derived growth factor (PDGF) G. A. A. Ferns, et al., “Inhibition of Neoinitmal Smooth Muscle Accumulation after Angioplasty by an Antibody to PDGF,” Science, Vol. 253, pp. 1129-1132 (1991), basic fibroblast growth factor (bFGF) V. Lindner, et al., “Role of Basic Fibroblast Growth Factor in Vascular Lesion Formation,” Circ. Res., Vol. 68, pp. 106-113 (1991), and interleukin-1 (IL-1) H. Loppnow and P. Libby, “Proliferating or Interleukin-1 Activated Human Vascular Smooth Muscle Cells Secrete Copious Interleukin 6,” J. Clin. Invest., Vol. 85, pp. 731-738 (1990).

Macrophages and platelets also release enzymes, i.e., elastase, collagenase, heparanase) that digest various constituents of the ECM and release bFGF and possibly other growth factors (TGFB) that are stored in basement membranes and ECM I. Vlodaysky, et al., “Extracellylar Matrix-bound Growth Factors, Enzymes and Plasma Proteins,” in: Molecular and Cellular Aspects of Basement Membranes, Monographs in Cell Biology, D. H. Rohrbach and R. Timpl, Eds., Academic Press, New York, N.Y., U.S.A., pp. 327-346 (1993). A potent growth-promoting activity towards SMCs is also exerted by thrombin, which, under certain conditions, may be present within the vessel wall R. Bar-Shavit, et al., “Thrombin Immobilized to Extracellular Matrix Is a Mitogen for Vascular Smooth Muscle Cells: Non-Enzymatic Mode of Action,” Cell Reg., Vol. 1, pp. 453-463 (1990); S. M. Schwartz, “Serum-Derived Growth Factor is Thrombin?” J. Clin. Invest., Vol 91, p. 4 (1993)!. Molecules that interfere with the growth-promoting activity of these growth factors may attenuate the progression of the atherogenic process.

Proliferation of arterial smooth muscle cells (SMC) in response to endothelial injury is a basic event in the process of restenosis of coronary arteries after percutaneous transluminal coronary angioplasty (PTCA) V. Fuster, et al., ibid. Coronary bypass surgery or angioplasty are applied to reopen coronary arteries that have been narrowed by heart disease. A major problem with both procedures is that arteries rapidly reclog in about 30% of patients undergoing angioplasty and about 10% of bypass surgery patients. Vascular SMC are ordinarily protected by the smooth inner lining of the arteries, composed of vascular endothelial cells. However, following bypass surgery or angioplasty, SMC are often left exposed. In a futile effort to repair the wound, the cells proliferate and clog the artery.

As described in U.S. Pat. No. 5,891,879, which is incorporated by reference herein in its entirety, halofuginone has been shown to be effective in inhibiting the activity of various growth factors, including bFGF, and inhibits autocrine growth of vascular SMC and fibroblasts. It was also shown that halofuginone can be used to inhibit SMC proliferation, to provide an effective strategy for inhibiting the pathophysiology of arteriosclerosis, restenosis after coronary angioplasty, and neointimal proliferation in saphenous vein grafts.

### Angiogenesis

Pathological situations exist in which the control mechanisms that normally operate to restrict angiogenesis are broken down, and an uncontrolled growth of blood vessels is unleashed. The resultant, excessive neovascularization underlies a number of so-called “angiogenic diseases” [J. Folkman, “Angiogenesis in Cancer, Vascular, Rheumatoid and Other Diseases,” Nature Medicine, Vol. 1, pp. 27-31 (1995)]. One group of angiogenic diseases comprises retinopathies distinguished by excessive in growth of blood vessels into the retina, leading to obstruction of vision and eventually to blindness [J. Folkman, ibid. (1995)].

However, the most devastating disease in which unwarranted angiogenesis plays a crucial role is the progression and spread of solid tumors, e.g., metastasis. It is now well-accepted that once tumor take has occurred, every increase in tumor cell population must be preceded by an increase in new capillaries that converge on the tumor and supply the cells with oxygen and nutrients [J. Folkman. ibid. (1985); J. Folkman, “What Is the Evidence that Tumors Are Angiogenesis Dependent?” J. Natl. Cancer Inst., Vol. 82, pp. 4-6 (1989); N. Weidner, et al., “Tumor Angiogenesis Correlates with Metastasis in Invasive Prostate Carcinoma,” Amer. J. Pathol., Vol. 143, pp. 401-409 (1993)]. Tumors may thus remain harmless and confined to their tissue of origin, as long as an accompanying angiogenic program is prevented from being activated.

Since the angiogenesis-dependent step in tumor progression is shared by solid tumors of all ethiologies, the ability to inhibit tumor-associated angiogenesis is a promising approach in combatting cancer [M. S. O'Reilly, et al., “A Novel Angiogenesis Inhibitor that Mediates the Suppression of Metastases by a Lewis Lung Carcinoma,” Cell, Vol. 79, pp. 316-328 (1994)]. A substantial body of experimental evidence supports the hypothesis that tumor angiogenesis is fundamental for the growth and metastasis of solid tumors [J. Folkman, ibid. (1989); N. Weidner, et al., ibid. (1993): M. S. O'Reilly, et al., ibid. (1994); N. Weidner, et al., “Tumor Angiogenesis and Metastasis—Correlation in Invasive Breast Carcinoma,” N. Eng. J. Med., Vol. 324, pp. 1-8 (1991)]. Indeed, the majority of solid tumors are not even clinically detectable until after the occurrence of neovascularizartion, whose induction in solid tumors is mediated by one or more angiogenic factors [J. Folkman, ibid. (1987); J. Folkman and Y. Shing, ibid. (1992)]. Moreover, the ability to inhibit blood vessel proliferation and penetration into a given organ carries the potential of treating other diseases, which is of paramount medical importance.

Protracted angiogenesis is also observed in a variety of pathologic states, such as arthritis, psoriasis, diabetic retinopathy, chronic inflammation, scleroderma, hemangioma, retrolental fibroplasia and abnormal capillary proliferation in hemophiliac joints, prolonged menstruation and bleeding, and other disorders of the female reproductive system [J. Folkman, ibid. (1995); J. W. Miller, et al., “Vascular Endothelial Growth Factor/Vascular Permeability Factor Is Temporarily and Partially Correlated with Ocular Angiogenesis in a Primate Model,” J. Pathol., Vol. 145, pp. 574-584 (1994); A. P. Adamis, et al., “Increased Vascular Endothelial Growth Factor Levels in the Vitreous of Eyes with Proliferative Diabetic Retinopathy,” Amer. J. Ophthal., Vol. 118, pp. 445-450 (1994); K. Takahashi, et al., “Cellular Markers that Distinguish the Phases of Hemangioma during Infancy and Childhood,” J. Clin. Invest., Vol. 93, pp. 2357-2364 (1994); D. J. Peacock, et al., “Angiogenesis Inhibition Suppresses Collagen Arthritis,” J. Exp. Med., Vol. 175, pp. 1135-1138 (1992); B. J. Nickoloff, et al., “Aberrant Production of Interleukin-8 and Thrombospondin-1 by Psoriatic Keratinocytes Mediates Angiogenesis,” Amer. J. Pathol., Vol. 44, pp. 820-828 (1994); J. Folkman, “Angiogenesis in Female Reproductive Organs,” in: Steroid Hormones and Uterine Bleeding, N. J. Alexander and C. d'Arcangues, Eds., American Association for the Advancement of Science Press, Washington, D.C., U.S.A., pp. 144-158 (1992)].

In many of the above-mentioned abnormalities, unrestrained new capillary growth itself contributes to the disease process. For example, in arthritis, new capillaries may invade and destroy joint cartilege. In diabetes, new capillaries in the eye hemorrhage and cause blindness. It is also possible that certain developmental disorders, such as intestinal atresia, vascular malformations, and unilateral fasial atrophy, may be due to angiogenic abnormality [J. Folkman, ibid. (1995)].

As described in U.S. Pat. No. 6,090,814, which is incorporated by reference herein in its entirety, halofuginone has been shown to be an effective angiogenesis inhibitor in cell lines. Halofuginone was also shown to be a strong inhibitor of DNA synthesis in human leiomyosarcoma tumor cells at low concentrations, regardless of whether the cells are stimulated by serum or by a potent growth-promoting factor such as HB-EGF.

### Malaria

Malaria is an infectious disease caused by four protozoan parasites: Plasmodium falciparum; Plasmodium vivax; Plasmodium ovale; Plasmodium berghei; and Plasmodium malaria. These four parasites are typically transmitted by the bite of an infected female Anopheles mosquito. Malaria is a problem in many parts of the world and over the last few decades the malaria burden has steadily increased. An estimated 1 to 3 million people die every year from malaria—mostly children under the age of 5. This increase in malaria mortality is due in part to the fact that Plasmodium falciparum, the deadliest malaria parasite, has acquired resistance against most available antimalarial drugs.

As described in Jiang, S. et al. Antimicrobial Agents and Chemotherapy 49(3): 1169-1176, 2005, which is incorporated by reference in its entirety, halofuginone was shown to reduce parasitemias to undetectable levels and displayed curative effects in Plasmodium berghei-infected mice. Therefore, an oral dosage form comprising halofuginone, or a pharmaceutically acceptable salt thereof, e.g. halofuginone hydrobromide, can be used for treating Plasmodium related diseases, e.g., malaria. Accordingly, provided herein are methods for preventing or treating malaria in a subject in need of such treatment, which method comprises administering to the subject a therapeutically effective amount of an oral dosage form comprising halofuginone, or a pharmaceutically acceptable salt thereof, e.g. halofuginone hydrobromide.

### Autoimmune Diseases and Immune Related Disorders

Exemplary autoimmune diseases and immune related disorders include those of the respiratory tract, such as obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung, idiopathic interstitial pneumonia, antitussive activity, treatment of chronic cough associated with inflammatory conditions of the airways or iatrogenic induced cough. Exemplary autoimmune diseases and immune related disorders of the bone and joint arthritis including rheumatic, infectious, autoimmune, seronegative spondyloarthropathies (such as ankylosing spondylitis, psoriatic arthritis or Reiter's disease), Behcet's disease, Sjogren's syndrome or systemic sclerosis. Exemplary autoimmune diseases and immune related disorders of the skin and eyes include psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis. Exemplary autoimmune diseases and immune related diroders fo the GI tract include Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema). The compounds described herein can also be used to treat allograft rejection, for example, acute and chronic following, for example, transplantation of kidney, heart, liver, lung, bone marrow, skin or cornea; or chronic graft versus host disease. Other exemplary autoimmune diseases and immune related disorders include Alzheimer's disease, multiple sclerosis, atherosclerosis, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura or disorders of the menstrual cycle.

### Multiple Sclerosis

Multiple sclerosis (MS) is a neuromuscular disease characterized by focal inflammatory and autoimmune degeneration of cerebral white matter. White matter becomes inflamed, and inflammation is followed by destruction of myelin (forming “lesions” which are marked by an infiltration of numerous immune cells, especially T-cell lymphocytes and macrophages. MS can cause a slowing or complete block of nerve impulse transmission and, thus, diminished or lost bodily function. A patient who has MS may have one of a variety of grade of MS (e.g., relapsing-remitting MS, primary progressive MS, secondary progressive, and Marburg's variant MS). Symptoms can include vision problems such as blurred or double vision, redgreen color distortion, or even blindness in one eye, muscle weakness in the extremities, coordination and balance problems, muscle spasticity, muscle fatigue, paresthesias, fleeting abnormal sensory feelings such as numbness, prickling, or “pins and needles” sensations, and in the worst cases, partial or complete paralysis. About half of the people suffering from MS also experience cognitive impairments, such as for example, poor concentration, attention, memory and/or judgment. (see, e.g., US 5 2003-0130357 and 2003-0092089).

### Scleroderma

Scleroderma is a chronic systemic autoimmune disease (primarily of the skin—“derma”), and can be characterized by fibrosis (or hardening—“sclero”), vascular alterations, and autoantibodies. There are two major forms of Scleroderma:

Limited systemic sclerosis/scleroderma involves cutaneous manifestations that mainly affect the hands, arms, and face. It was previously called CREST syndrome in reference to the following complications: Calcinosis, Raynaud's phenomenon, Esophageal dysfunction, Sclerodactyly, and Telangiectasias. Additionally, pulmonary arterial hypertension may occur in up to one-third of patients and is the most serious complication for this form of scleroderma. Diffuse systemic sclerosis/scleroderma is rapidly progressing and affects a large area of the skin and one or more internal organs, frequently the kidneys, esophagus, heart, and lungs. This form of scleroderma can be quite disabling. There are no treatments for scleroderma itself, but individual organ system complications are treated. Other forms of scleroderma include systemic sine scleroderma (which lacks skin changes but has systemic manifestations) and two localized forms, morphea and linear scleroderma, which affect the skin but not the internal organs.

### EXAMPLES

The disclosure is further described in the following examples, which do not limit the scope of the claims.

### Example A—Release Profile of Coated and Uncoated Halofuginone Tablets

Halofuginone (7-Bromo-6-chloro-3-[3-[(2S,3R)-3-hydroxy-2-piperidinyl]-2-oxopropyl]-4-quinazolinone) hydrobromide was blended with lactose at 2% (wt/wt) and hand filled into size 4 capsules, approximately 50 mg (non-enteric capsules). Test capsules were evaluated using HPLC and found to contain between 0.98 and 1.02 g of halofuginone hydrobromide. Some non-enteric capsules were coated with an enteric coating (below), while others will be used as a control against the enteric-coated capsules.

Halofuginone hydrobromide non-enteric capsules were coated in a pan coater with a hydroxypropyl methylcellulose (HPMC) undercoat (to ensure tablet closure) followed by a poly(methacrylic acid-co-ethyl acrylate) enteric coating (Eudragit L30 D-55, Evonik, Essen, Germany). The dissolution properties of the enteric-coated and the non-enteric coated capsules were then evaluated in simulated gastric conditions and simulated intestinal conditions at 37° C. [See, Dressman, “Dissolution Media Simulating Conditions in the Proximal Human Gastrointestinal Tract: An Update,” Pharmaceutical Research, vol. 25, 1663-1676 (2008), for simulated formulations.] Three tablets were evaluated for each combination, e.g., three enteric-coated tablets in simulated gastric conditions, three non-enteric coated tablets in simulated gastric conditions, etc. Each tablet was placed in the respective fluid and then weighed at several time points to produce a dissolution profile as a function of time.

TABLE A1
Average dissolution time in simulated gastric environment.

| Time in solution | Enteric-coated capsule | Non-enteric-coated capsule |
|---|---|---|
| (min) | (% dissolved) | (% dissolved) |
| 15 | 0 | 94.074 |
| 30 | 0 | 96.134 |
| 45 | 0 | 96.945 |
| 60 | 0 | 97.812 |

TABLE A2
Average dissolution time in simulated intestinal environment.

| Time in solution | Enteric-coated capsule | Non-enteric-coated capsule |
|---|---|---|
| (min) | (% dissolved) | (% dissolved) |
| 30 | 4.107 | 85.582 |
| 60 | 70.833 | 92.052 |
| 90 | 91.231 | 91.42 |
| 120 | 90.618 | 92.183 |
| 180 | 90.056 | 91.893 |
| 210 | 91.618 | 95.014 |

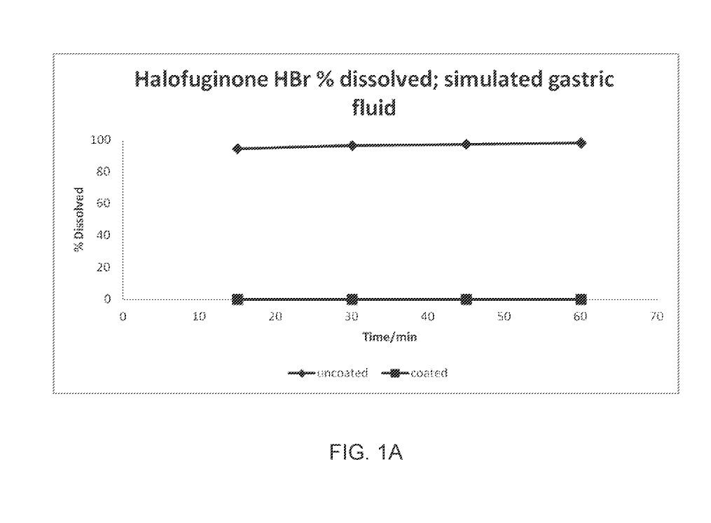

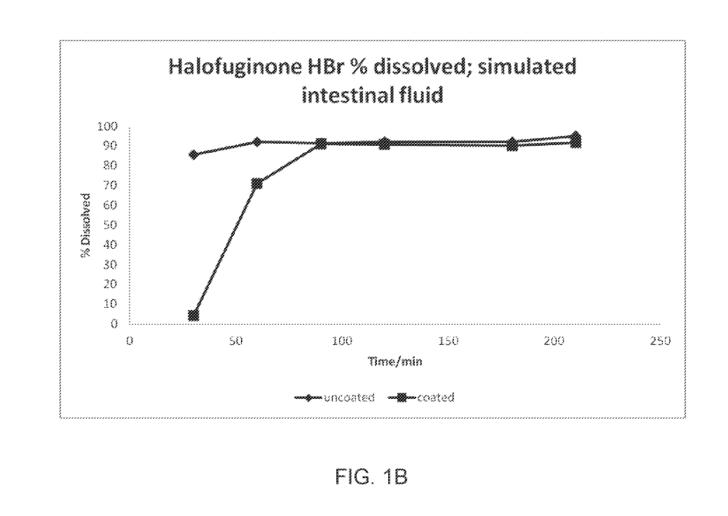

As shown in Tables A1 and A2 and FIG. 1A, the enteric coated capsules did not dissolve in simulated gastric conditions, while the non-enteric coated capsules dissolved in the gastric conditions. Additionally, as shown in FIG. 1B, the enteric coated capsule dissolved in the simulated intestinal conditions as did the non-enteric coated capsule.

### Example B—Administration of Enteric-Coated Halofuginone Hydrobromide in Beagle Dogs

Five groups of five male beagle dogs (25 total dogs) were orally administered various compositions of halofuginone solutions. One group of five was administered a clear 0.15 mg/kg aqueous solution comprising lactic acid; one group was administered non-enteric-coated capsules of Example A at 0.10 mg/kg; one group was administered non-enteric-coated capsules of Example A at 0.15 mg/kg; one group was administered enteric-coated capsules of Example A at 0.10 mg/kg; one group was administered enteric-coated capsules of Example A at 0.15 mg/kg. The dogs were fasted overnight prior to administration, and food was returned four hours post-administration. Plasma was drawn from each animal at the time of administration, and then at eight successive time points (0.5, 1, 2, 4, 8, 12, 24, and 48 hours post-administration). Additionally, each dog was observed for 48 hours and any physiological changes were noted. As can be seen by comparing Tables B2 and B4 to Tables B3 and B5, the cohorts that were administered the enteric-coated capsules had fewer digestive problems, and less severe digestive problems, than the cohorts that were administered the non-enteric-coated capsules.

TABLE B1
Observations of beagle dogs orally administered
0.15 mg/kg halofuginone as aqueous solution.

| | Time of | |
|---|---|---|
| Animal | Observation | |
| # | (h postdose) | Clinical Observation |
| B1-1 | ~0.75 | Animal produced white foamy emesis. |
| | ~1 | Animal had white foamy emesis. |
| | ~1.5 | Animal produced 2 instances of white |
| | | foamy emesis. |
| | 24 | Animal was observed to have a red tint |
| | | to its urine. |
| | 48 | Animal's urine was normal in color. |
| B1-2 | Following 0.25 | Animal produced clear foamy emesis. |
| | Prior to 0.75 | Animal produced 2 episodes of white |
| | | foamy emesis. |
| | ~0.75 | Animal produced white foamy emesis. |
| | Prior to 1 | Animal had white foamy emesis. |
| | ~1 | Animal had white foamy emesis. |
| | ~1.25 | Animal had clear to white foamy emesis. |
| | Prior to 1.5 | Animal produced white foamy emesis. |
| | ~1.5 | Animal had white foamy emesis. |
| B1-3 | Prior to 0.5 | Animal had 2 instances of foamy clear |
| | | emesis under home cage. |
| | Prior to 0.75 | Animal produced 2 episodes of white |
| | | foamy emesis. |
| | ~0.75 | Animal produced white foamy emesis. |
| | ~1.25 | Animal had clear to white foamy emesis. |
| | ~1.5 | Animal produced white foamy emesis. |
| | Prior to 2 | Animal produced white foamy emesis. |
| | 2 | Animal had reduced activity and showed |
| | | some trembling. |
| B1-4 | Following 0.5 | Animal produced white foamy emesis. |
| | ~0.75 | Animal had white foamy emesis. |
| | ~1 | Animal had white foamy emesis. |
| | ~1.25 | Animal had clear to white foamy emesis. |
| | Prior to 1.5 | Animal produced white foamy emesis. |
| B1-5 | Following 0.75 | Animal had white foamy emesis. |
| | Following 1 | Animal had clear to white foamy emesis. |
| | ~1.5 | Animal produced white foamy emesis. |
| | 2 | Animal showed an increase in salivation. |
| | 8 | Animal had soft mucoidal stool along with |
| | | normal stool. |

TABLE B2
Observations of beagle dogs orally administered 0.10
mg/kg halofuginone in non-enteric coated capsule.

| Animal | Time of | |
|---|---|---|
| # | Observation | Clinical Observation |
| B2-1 | ~1.25 | Animal had a large amount of yellow foamy |
| | | emesis. |
| | ~1.75 | Animal had white foamy emesis. |
| | 12 | Animal had white foamy emesis. |
| B2-2 | ~1 min | Animal had white foamy emesis that |
| | following dose | contained a partial capsule that was |
| | administration | unrecoverable. |
| | Prior to 0.5 | Animal had a small amount of white foamy |
| | | emesis. |
| B2-3 | Prior to 0.5 | Animal had a small amount of white foamy |
| | | emesis. |
| | Prior to 0.75 | Animal had a large amount of white foamy |
| | | emesis. |
| | Following 0.75 | Animal had white foamy emesis. |
| | Prior to 1 | Animal had white foamy emesis. |
| B2-4 | ~1.25 | Animal had yellow foamy emesis. |
| | ~1.5 | Animal had 2 occurances of white foamy |
| | | emesis. |
| B2-5 | ~1.75 | Animal had yellow foamy emesis. |

TABLE B3
Observations of beagle dogs orally administered 0.10
mg/kg halofuginone in enteric coated capsule.

| | Time of | |
|---|---|---|
| Animal | Observation | |
| # | (h postdose) | Clinical Observation |
| B3-1 | Prior to 2 | Animal had a small amount of clear to white |
| | | foamy emesis. |
| | ~7 | Animal had 2 occurances of yellow foamy |
| | | emesis. |
| B3-2 | 24 | Animal has a small amount of loose stool. |
| | 48 | Animal had a small amount of partially |
| | | digested food emesis. |
| B3-3 | N/A | No unusal observations were noted for Week 3. |
| B3-4 | Predose | Animal had 5 occurances of emesis. (White |
| | | foamy emesis or partially digested food |
| | | emesis.) |
| B3-5 | 24 | Animal had a small amount of partially |
| | | digested food emesis. |
| | 1 | Animal had a small amount of mucoidal stool. |
| | 4 | Animal had a small amount of yellow emesis. |

TABLE B4
Observations of beagle dogs orally administered 0.20
mg/kg halofuginone in non-enteric coated capsule.

| | Time of | |
|---|---|---|
| Animal | Observation | |
| # | (h postdose) | Clinical Observation |
| B4-1 | N/A | No unusal observations noted for |
| | | Week 2. |
| B4-2 | Following 1.75 | Animal had white foamy emesis. |
| | 12 | Animal had loose mucoidal stool. |
| B4-3 | Prior to 0.5 | Animal had white foamy emesis. |
| | Following the 1.25 | Animal had white foamy emesis. |
| | ~1.75 | Animal had white foamy emesis. |
| B4-4 | Prior to 0.5 | Animal had white foamy emesis. |
| | Following 0.5 | Animal had white foamy emesis. |
| | Following 0.75 | Animal had white foamy emesis. |
| | Following 1.25 | Animal had white foamy emesis. |
| | ~2 | Animal had white foamy emesis. |
| | 4 | Animal had white foamy emesis. |
| B4-5 | Prior to 0.5 | Animal had white foamy emesis. |
| | Prior to 0.75 | Animal had white foamy emesis. |
| | Prior to 1 | Animal had yellow foamy emesis. |
| | ~1.25 | Animal had white foamy emesis. |
| | Following 1.25 | Animal had white foamy emesis. |
| | Following 1.5 | Animal had 2 occurances of white |
| | | foamy emesis. |
| | ~2 | Animal had white foamy emesis. |
| | 4 | Animal had white foamy emesis. |

TABLE B5
Observations of beagle dogs orally administered 0.20
mg/kg halofuginone in enteric coated capsule.

| | Time of | |
|---|---|---|
| Animal | Observation | |
| # | (h postdose) | Clinical Observation |
| B5-1 | N/A | No unusal observations were noted for Week 3. |
| B5-2 | N/A | No unusal observations were noted for Week 3. |
| B5-3 | 4 | Animal had white foamy emesis. |
| B5-4 | Immediely | Animal had white foamy emesis containing |
| | following dose | both capsules. Capsules were recovered, |
| | administration. | animal was re-dosed at this time. |
| | Following 1.5 | Animal had 2 occurances of white foamy |
| | | emesis. |
| | Prior to 2 | Animal had 4 occurances of foamy white |
| | | emesis. |
| | 4 | Animal had white foamy emesis. |
| B5-5 | 4 | Animal had white foamy emesis. |
| | 24 | Animal has a small amount of soft to loose |
| | | stool. |

### Plasma Analysis

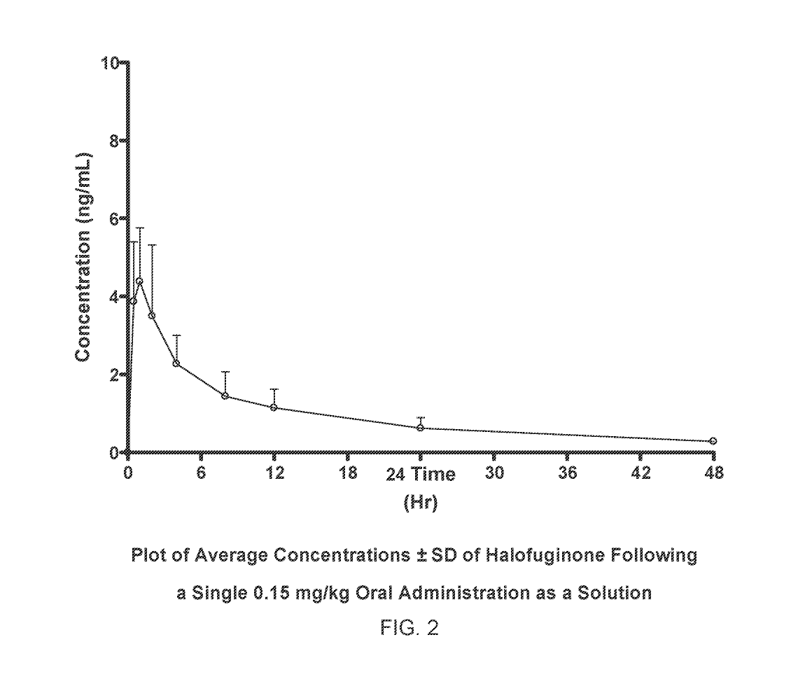

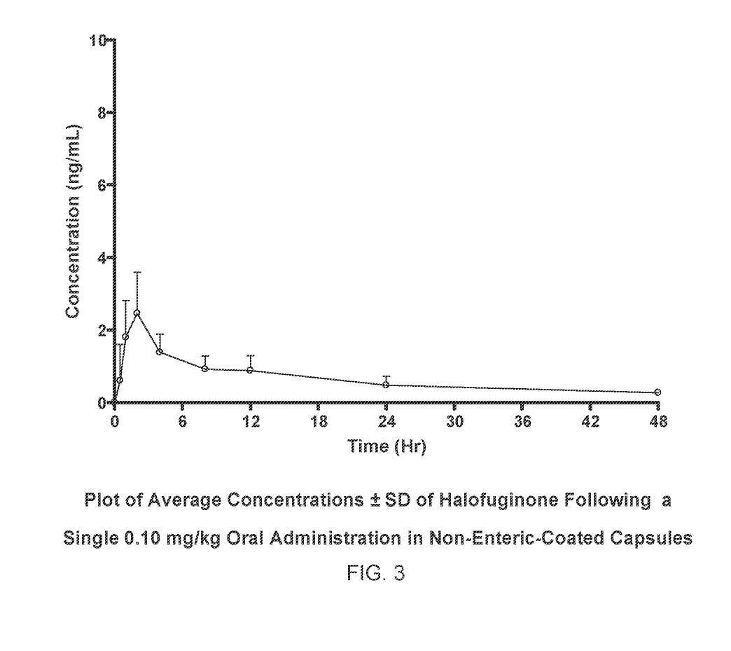

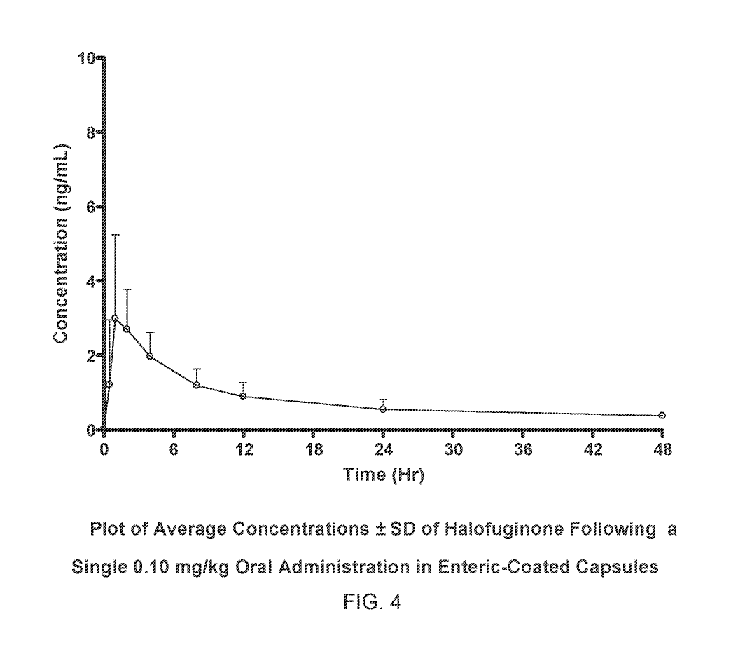

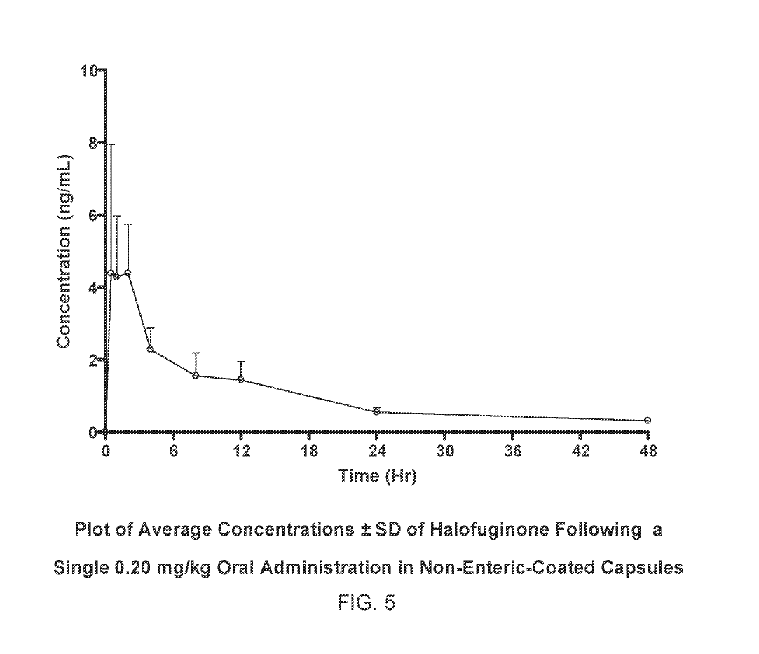

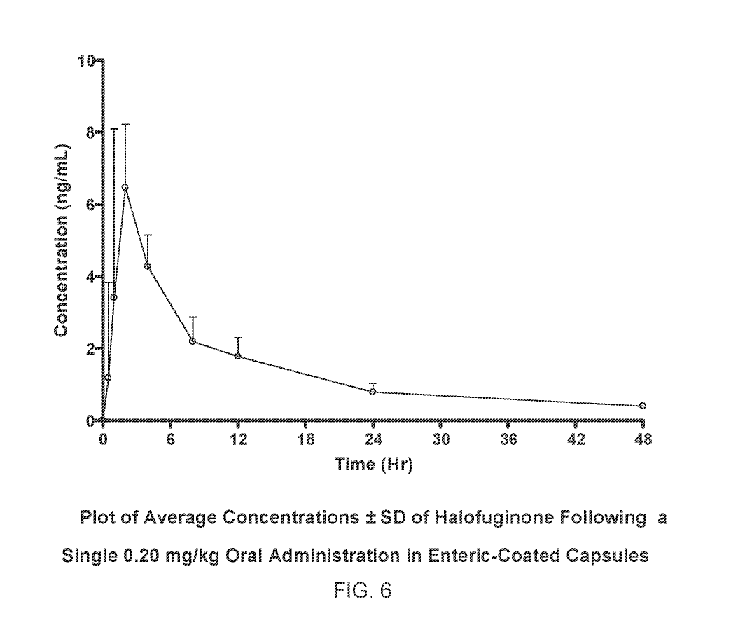

The plasma samples, collected from the dogs at the various time points, were assayed for halofuginone concentrations using HPLC-MS/MS (ACE Excel 2C18 AR, 50×2.1 mm column). The averaged results of the assays for each dosage regimen are shown in FIGS. 2-6. The averaged results of several pharmacokinetic parameters for each dosage regiment are shown in TABLES B6 and B7. [Cₘₐₓ: maximum observed halofuginone concentration; tₘₐₓ: time point at Cₘₐₓ; AUC₍₀₋ₜ₎: AUC to the last non-zero concentration (t is the corresponding time); AUC_{(0-∞)}: AUC_{(0-∞)}=AUC₍₀₋ₜ₎+AUC_{(t-∞)}; t_{1/2}: half-life; time taken for drug plasma concentration to fall by one-half; Vz_obs: observed volume of distribution; Cl_obs: observed clearance]

TABLE B6
Pharmacokinetic parameters of halofuginone in the plasma
of male beagle dogs following administration of 0.15 mg/kg
of aqueous solution, 0.10 mg/kg of non-enteric-coated
capsules, or 0.10 mg/kg of enteric-coated capsules.

| | | | Non-Enteric Coated | | Enteric Coated | |
|---|---|---|---|---|---|---|
| | Clear Solution | | Capsule | | Capsules | |
| Parameter | 0.15 mg/kg | | 0.10 mg/kg | | 0.10 mg/kg | |
| (units) | Mean | SD | Mean | SD | Mean | SD |
| Cₘₐₓ | 5.26 | 1.10 | 2.54 | 1.14 | 3.76 | 1.53 |
| (ng/mL) | | | | | | |
| tₘₐₓ (hr) | 0.90 | 0.65 | 1.50 | 0.58 | 1.20 | 0.45 |
| AUC₍₀₋ₜ₎ | 39.50 | 14.00 | 25.47 | 12.50 | 33.45 | 16.91 |
| (ng · hr/mL) | | | | | | |
| AUC_{(0-∞)} | 49.08 | 17.87 | 37.53 | 18.69 | 43.58 | 18.25 |
| (ng · hr/mL) | | | | | | |
| t_{1/2} (hr) | 14.82 | 4.55 | 18.96 | 8.01 | 18.46 | 7.70 |
| Vz_obs | 70.73 | 24.54 | 81.72 | 48.89 | 62.35 | 30.20 |
| (L/kg) | | | | | | |
| Cl_obs | 3.47 | 1.51 | 3.14 | 1.51 | 2.51 | 0.96 |
| (L/hr/kg) | | | | | | |

TABLE B7
Pharmacokinetic parameters of halofuginone in the plasma of male
beagle dogs following administration of 0.20 mg/kg of non-enteric-
coated capsules or 0.20 mg/kg of enteric-coated capsules.

| | | |
|---|---|---|
| | Non-Enteric Coated | Enteric Coated |
| | Capsule | Capsules |
| Parameter | 0.20 mg/kg | 0.20 mg/kg |

| (units) | Mean | SD | Mean | SD |
|---|---|---|---|---|
| Cₘₐₓ | 5.64 | 2.42 | 7.18 | 2.14 |
| (ng/mL) | | | | |
| tₘₐₓ (hr) | 1.10 | 0.82 | 1.60 | 0.55 |
| AUC₍₀₋ₜ₎ | 42.88 | 15.66 | 63.43 | 23.27 |
| (ng · hr/mL) | | | | |
| AUC_{(0-∞)} | 51.06 | 15.65 | 72.18 | 23.86 |
| (ng · hr/mL) | | | | |
| t_{1/2} (hr) | 13.62 | 3.83 | 13.58 | 2.88 |
| Vz_obs | 78.51 | 23.64 | 57.08 | 21.74 |
| (L/kg) | | | | |
| Cl_obs | 4.09 | 1.25 | 3.06 | 1.49 |
| (L/hr/kg) | | | | |

As shown in Table B6 and B7, above, the average Cₘₐₓ, for the enteric-coated capsules was higher than the Cmax for the non-enteric-coated capsules for the same administration of halofuginone. Presumably, the difference is due to the location of the absorption (stomach vs. intestine) and/or the undesired destruction of halofuginone in the lower pH environment of the stomach.

### Example C—Administration of Oral and Parenteral Forms of Halofuginone

### Study Design

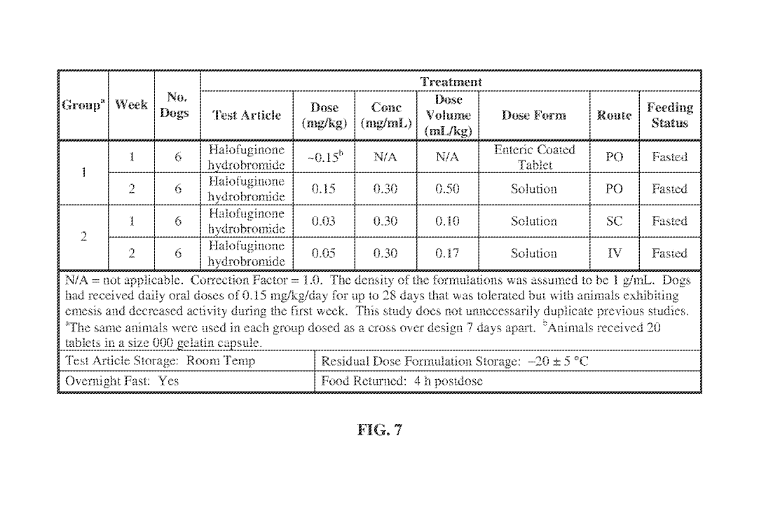

Two groups of 6 male Beagle dogs were dosed with two formulations or two routes of administration of Halofuginone hydrobromide. In a cross-over study design, one group received an oral (PO) dose at ˜0.15 mg/kg as enteric-coated (EC) tablets in a gelatin capsule and then another oral dose at 0.15 mg/kg as a solution 7 days later. The second group received a subcutaneous (SC) dose at 0.03 mg/kg as a solution and then an intravenous (IV) dose at 0.05 mg/kg as a solution. The test article was administered once for each dose level and dosage form. The animals were dosed once a week or after completing at least a 7 day washout period. The dose formulations of Halofuginone hydrobromide were EC tablets or 0.3 mg/mL solutions. The EC tablets contained 0.075 mg of Halofuginone hydrobromide. The tablets (20/dog) were loaded into a gelatin capsule prior to dosing. This study was conducted to confirm that the clinical tablet formulation has similar gastrointestinal (GI) sparing characteristics as the hand packed experimental capsules used in a previous study and to determine the absolute bioavailability of these formulations. Dogs were selected because prior GLP-compliant toxicology results suggested this was the most sensitive species in which to observe the expected adverse GI effects. Study Design is summarized in FIG. 7.

FIG. 7. Study Design

| | | | Treatme | | | | | | |
|---|---|---|---|---|---|---|---|---|---|
| | | | | | Conc | | | | |
| Group | | No. | Test | Dose | | Dose | | | Feeding |
| a | Week | Dogs | Article | (mg/kg) | (mg/mL) | Volume | Dose Form | Route | Status |
| 1 | 1 | 6 | Halofuginone | ~0.15^{b} | N/A | N/A | Enteric | PO | Fasted |
| | | | | | | | Coated | | |
| | 2 | 6 | Halofuginone | 0.15 | 0.30 | 0.50 | Solution | PO | Fasted |
| 2 | 1 | 6 | Halofuginone | 0.03 | 0.30 | 0.10 | Solution | SC | Fasted |
| | 2 | 6 | Halofuginone | 0.05 | 0.30 | 0.17 | Solution | IV | Fasted |

N/A = not applicable.
Correction Factor = 1.0.
The density of the formulations was assumed to be 1 g/mL.
Dogs had received daily oral doses of 0.15 mg/kg/day for up to 28 days that was tolerated but with animals
exhibiting emesis and decreased activity during the first week. This study does not unnecessarily duplicate previous studies.

| | |
|---|---|
| Test Article Storage: Room Temp | Residual Dose Formulation Storage: □20 ± 5 □C. |
| Overnight Fast: Yes | Food Returned: 4 h postdose |

The dose levels were selected based on the results of a previous study in dogs in which the high-dose group was dosed at 0.15 mg/kg/day for up to 28 days (this dose was tolerated for the entire course of the study). Clinical signs of toxicity in that study included emesis and decreased activity during the first week of dosing.

### Clinical Observations

Each animal received a physical examination prior to dosing, with cage side observations for mortality and morbidity twice daily thereafter. Detailed clinical observations were performed prior to dosing and at 15 minute (min) intervals during the first 2 hours (h) postdose, at 4, 8, and 24 h postdose on dosing days. Observations included, but were not limited to, monitoring for emesis and gastrointestinal effects, evaluation of the skin, fur, eyes, ears, nose, oral cavity, thorax, abdomen, external genitalia, limbs and feet, respiratory and circulatory effects, autonomic effects such as salivation, nervous system effects including tremors, convulsions, reactivity to handling, and unusual behavior. Body weights were obtained on the day prior to dosing to determine dosing solution volume. See Tables C11-C14 for a complete list of the clinical observations.

TABLE C11
Clinical Observations in Beagle Dogs Following a Single
0.15 mg/kg Oral Administration as Enteric-Coated Tablets

| | | | Time | |
|---|---|---|---|---|
| | | Animal | of | Clinical |
| Group | Week | # | Observation | Observation |
| 1 | 1 | 1 | 12 | Loose stool |
| | | 2 | 12 | Loose stool |
| | | 3 | 1.75 | Yellow, foamy emesis |
| | | | 2 | Soft stool and three |
| | | | | separate episodes of |
| | | | | yellow, foamy emesis in |
| | | 4 | 8 | Loose stool |
| | | | 12 | Mucoidal stool |
| | | 5 | 1 | Yellow, foamy emesis |
| | | | 1.25 | Yellow, foamy emesis |
| | | 6 | 4 | Loose stool |
| | | | 24 | Loose/mucoidal stool |

TABLE C12
Clinical Observations in Beagle Dogs Following a Single
0.15 mg/kg Oral Administration as a Clear Solution

| | | | Time | |
|---|---|---|---|---|
| | | Animal | of | Clinical |
| Group | Week | # | Observation | Obsevation |
| 1 | 2 | 1 | 0.4 | Foamy, white emesis |
| | | | 12 | Soft Stool |
| | | | 48 | Soft Stool |
| | | 2 | 12 | Soft Stool |
| | | 3 | 0.33-1 | Eight separate episodes |
| | | | | of emesis, all white foam |
| | | | 2 | Lethargic and was shaking |
| | | 4 | 0.25 | Foamy, white emesis |
| | | 5 | 0.5 | Foamy, white emesis |
| | | | 0.66 | Foamy, white emesis |
| | | | 0.9 | Foamy, white emesis |
| | | | 1.66 | Loose stool |
| | | 6 | 0.25 | Foamy, white emesis |
| | | | 0.5 | Foamy, white emesis |
| | | | 0.66 | Foamy, white emesis |
| | | | 0.9 | Foamy, white emesis |
| | | | 1 | Foamy, white emesis |

TABLE C13
Clinical Observations in Beagle Dogs Following a Single 0.03
mg/kg Subcutaneous Administration as a Clear Solution

| | | | Time | |
|---|---|---|---|---|
| | | Animal | of | Clinical |
| Group | Week | # | Observation | Observation |
| 2 | 1 | 7 | — | — |
| | | 8 | — | — |
| | | 9 | — | — |
| | | 10 | — | — |
| | | 11 | — | — |
| | | 12 | — | — |

— = not applicable

TABLE C14
Clinical Observations in Beagle Dogs Following a Single
0.05 mg/kg Intravenous Administration as a Clear Solution

| | | | Time | |
|---|---|---|---|---|
| | | Animal | of | Clinical |
| Group | Week | # | Observation | Observation |
| 2 | 2 | 7 | — | — |
| | | 8 | — | — |
| | | 9 | 8 | Soft stool |
| | | 10 | 12 | Soft Stool |
| | | 11 | — | — |
| | | 12 | 48 | Soft Stool |

— = not applicable

### Pharmacokinetic Analysis

Blood samples were collected from the jugular, cephalic, or saphenous veins into tubes containing EDTA at the following time points: 0, 0.5, 1, 2, 4, 8, 12, 24, and 48 h postdose. Blood samples were kept on wet ice until processing. Blood samples were centrifuged at 3200 RPM for 10 min at ˜5° C. Plasma samples were directly transferred to a 96-well plate tube (1.1 mL). Plug caps were placed on the tubes. Plasma samples were stored at □20±5° C. until they were shipped for analysis.

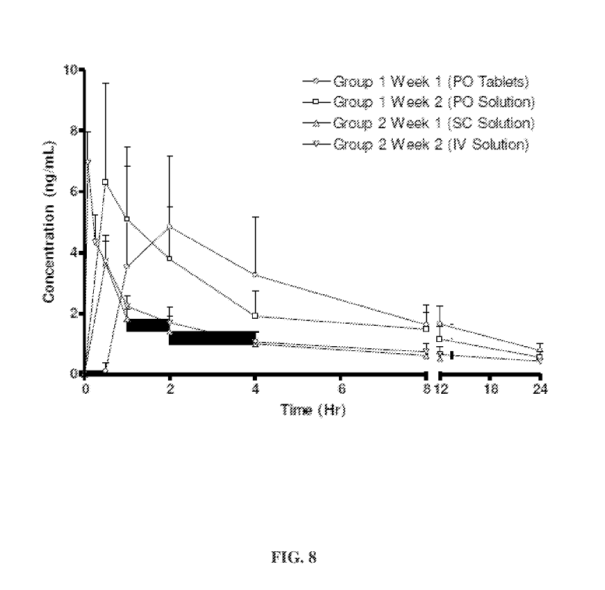

Individual and mean concentrations of Halofuginone hydrobromide in the plasma can be found in Tables C1-C4 and graphically in FIG. 8. Mean pharmacokinetic parameters can be found in Table C5 and Table C6 with individual pharmacokinetic parameters in Tables C7-C10.

TABLE C1
Individual Beagle Dog Plasma Concentrations of Halofuginone hydrobromide Following
a Single 0.15 mg/kg Oral Administration as Enteric-Coated Tablets

| Dog | 1 | 2 | 3 | 4 | 5 | 6 |
|---|---|---|---|---|---|---|
| Animal Weight | 10.540 | 8.383 | 8.470 | 8.528 | 8.963 | 9.101 |
| Animal Dose | 1.50 | 1.50 | 1.50 | 1.50 | 1.50 | 1.50 |
| Actual Dose | 0.14 | 0.18 | 0.18 | 0.18 | 0.17 | 0.16 |

| | | | | | | | Mean | SD |
|---|---|---|---|---|---|---|---|---|
| Time | Sample | Sample | Sample | Sample | Sample | Sample | Conc. | Conc. |
| (h) | Conc. | Conc. | Conc. | Conc. | Conc. | Conc. | (ng/m | (ng/mL |
| 0 | BLQ | BLQ | BLQ | BLQ | BLQ | BLQ | N/A | N/A |
| 0.5 | BLQ | BLQ | BLQ | BLQ | 0.68 | BLQ | 0.68 | N/A |
| 1 | 2.56 | 4.91 | 0.14 | 5.42 | 8.34 | BLQ | 4.28 | 3.09 |
| 2 | 1.93 | 4.35 | 8.28 | 2.85 | 5.35 | 6.31 | 4.55 | 2.47 |
| 4 | 1.19 | 2.96 | 6.44 | 1.86 | 2.69 | 4.42 | 3.03 | 2.03 |
| 8 | 0.81 | 1.67 | 2.49 | 0.89 | 1.99 | 1.94 | 1.57 | 0.72 |
| 12 | 2.37 | 1.67 | 1.97 | 0.66 | 1.47 | 1.78 | 1.63 | 0.64 |
| 24 | 0.89 | 0.78 | 1.16 | 0.52 | 0.64 | 0.74 | 0.80 | 0.25 |
| 48 | 0.36 | 0.27 | 0.44 | 0.12 | 0.22 | 0.29 | 0.28 | 0.13 |

BLQ = Below Limit of Quantitation
N/A = not applicable

TABLE C2
Individual Beagle Dog Plasma Concentrations of Halofuginone hydrobromide
Following a Single 0.15 mg/kg Oral Administration as a Clear Solution

| Dog | 1 | 2 | 3 | 4 | 5 | 6 |
|---|---|---|---|---|---|---|
| Conc (mg/mL) | 0.3 | 0.3 | 0.3 | 0.3 | 0.3 | 0.3 |
| Animal Weight | 10.712 | 8.646 | 8.332 | 8.454 | 8.970 | 9.107 |
| Animal Dose | 5.36 | 4.32 | 4.17 | 4.23 | 4.49 | 4.55 |
| Actual Dose | 0.15 | 0.15 | 0.15 | 0.15 | 0.15 | 0.15 |

| | Sample | Sample | Sample | Sample | Sample | Sample | Mean | |
|---|---|---|---|---|---|---|---|---|
| Time | Conc. | Conc. | Conc. | Conc. | Conc. | Conc. | Conc. | SD |
| (h) | (ng/m | (ng/m | (ng/m | (ng/m | (ng/m | (ng/m | (ng/m | Conc. |
| 0 | BLQ | BLQ | BLQ | BLQ | BLQ | BLQ | N/A | N/A |
| 0.5 | 11.57 | 2.46 | 3.78 | 6.05 | 8.15 | 5.87 | 6.41 | 3.62 |
| 1 | 8.28 | 3.83 | 2.18 | 3.36 | 7.20 | 5.67 | 4.97 | 2.63 |
| 2 | 6.54 | 3.38 | 1.84 | 2.30 | 4.37 | 4.32 | 3.68 | 1.87 |
| 4 | 3.10 | 2.23 | 0.89 | 1.00 | 2.14 | 2.11 | 1.87 | 0.92 |
| 8 | 1.86 | 1.66 | 0.70 | 0.82 | 1.84 | 2.02 | 1.38 | 0.57 |
| 12 | 1.76 | 1.30 | 0.58 | 0.81 | 1.26 | 1.22 | 1.14 | 0.46 |
| 24 | 0.82 | 0.58 | 0.35 | 0.34 | 0.54 | 0.74 | 0.53 | 0.20 |
| 48 | 0.25 | 0.25 | 0.10 | BLQ | 0.18 | 0.20 | 0.20 | 0.07 |

BLQ = Below Limit of Quantitation
N/A = not applicable

TABLE C3
Individual Beagle Dog Plasma Concentrations of Halofuginone hydrobromide Following
a Single 0.03 mg/kg Subcutaneous Administration as a Clear Solution

| Dog | 7 | 8 | 9 | 10 | 11 | 12 |
|---|---|---|---|---|---|---|
| Conc (mg/mL) | 0.3 | 0.3 | 0.3 | 0.3 | 0.3 | 0.3 |
| Animal Weight | 8.924 | 8.512 | 9.244 | 8.651 | 9.391 | 8.724 |
| Animal Dose | 0.89 | 0.85 | 0.92 | 0.87 | 0.94 | 0.87 |
| Actual Dose | 0.03 | 0.03 | 0.03 | 0.03 | 0.03 | 0.03 |

| | Sample | Sample | Sample | Sample | Sample | Sample | Mean | |
|---|---|---|---|---|---|---|---|---|
| Time | Conc. | Conc. | Conc. | Conc. | Conc. | Conc. | Conc. | SD |
| (h) | (ng/m | (ng/m | (ng/m | (ng/m | (ng/m | (ng/m | (ng/m | Conc. |
| 0 | BLQ | BLQ | BLQ | BLQ | BLQ | BLQ | N/A | N/A |
| 0.5 | 2.82 | 2.95 | 2.98 | 5.07 | 4.29 | 3.82 | 3.62 | 1.01 |
| 1 | 1.67 | 1.62 | 1.37 | 2.04 | 2.27 | 1.98 | 1.79 | 0.36 |
| 2 | 0.90 | 0.85 | 1.28 | 1.95 | 2.03 | 1.46 | 1.40 | 0.56 |
| 4 | 0.88 | 0.70 | 0.58 | 1.56 | 1.29 | 1.02 | 1.00 | 0.41 |
| 8 | 0.46 | 0.51 | 0.39 | 0.92 | 0.86 | 0.58 | 0.63 | 0.24 |
| 12 | 0.39 | 0.38 | 0.38 | 0.78 | 0.63 | 0.58 | 0.51 | 0.19 |
| 24 | 0.11 | BLQ | 0.21 | 0.24 | 0.14 | 0.16 | 0.14 | 0.03 |
| 48 | BLQ | BLQ | BLQ | 0.16 | 0.12 | BLQ | N/A | N/A |

BLQ = Below Limit of Quantitation
N/A = not applicable

TABLE C4
Individual Beagle Dog Plasma Concentrations of Halofuginone hydrobromide Following
a Single 0.05 mg/kg Intravenous Administration as a Clear Solution

| Dog | 7 | 8 | 9 | 10 | 11 | 12 |
|---|---|---|---|---|---|---|
| Conc (mg/mL) | 0.3 | 0.3 | 0.3 | 0.3 | 0.3 | 0.3 |
| Animal Weight | 9.205 | 8.888 | 9.445 | 8.673 | 9.470 | 8.850 |
| Animal Dose | 1.56 | 1.51 | 1.61 | 1.47 | 1.61 | 1.50 |
| Actual Dose | 0.05 | 0.05 | 0.05 | 0.05 | 0.05 | 0.05 |

| | Sample | Sample | Sample | Sample | Sample | Sample | Mean | |
|---|---|---|---|---|---|---|---|---|
| Time | Conc. | Conc. | Conc. | Conc. | Conc. | Conc. | Conc. | SD |
| (h) | (ng/m | (ng/m | (ng/m | (ng/m | (ng/m | (ng/m | (ng/m | Conc. |
| 0 | BLQ | BLQ | BLQ | BLQ | BLQ | BLQ | N/A | N/A |
| 0.083 | 7.81 | 6.64 | 5.12 | 7.42 | 7.80 | 6.88 | 6.96 | 1.13 |
| 0.5 | 5.52 | 4.15 | 3.22 | 4.30 | 5.18 | 3.67 | 4.47 | 0.91 |
| 1 | 3.55 | 3.47 | 3.03 | 4.20 | 4.81 | 3.16 | 3.81 | 0.70 |
| 2 | 2.13 | 1.89 | 2.11 | 2.79 | 2.54 | 1.98 | 2.29 | 0.36 |
| 4 | 1.47 | 1.22 | 1.35 | 2.45 | 2.20 | 1.48 | 1.74 | 0.55 |
| 8 | 0.80 | 0.80 | 0.84 | 1.60 | 1.35 | 1.01 | 1.08 | 0.37 |
| 12 | 0.65 | 0.63 | 0.49 | 1.23 | 0.80 | 0.67 | 0.76 | 0.29 |
| 24 | 0.51 | 0.43 | 0.53 | 1.13 | 0.76 | 0.55 | 0.67 | 0.28 |
| 48 | 0.16 | 0.16 | 0.21 | 0.43 | 0.19 | 0.22 | 0.23 | 0.11 |

BLQ = Below Limit of Quantitation
N/A = not applicable

TABLE C5
Summary Pharmacokinetic Parameters of Halofuginone hydrobromide
in the Plasma of Beagle Dogs Following a 0.15 mg/kg Solution or
Capsule PO Administration
Group

| | | |
|---|---|---|
| | Week 1 | |
| | Enteric Coated | Week 2 |
| Parameter | Tablets in a Gelatin | Oral |

| (units) | Mean | SD | Mean | SD |
|---|---|---|---|---|
| Cₘₐₓ | 5.97 | 2.20 | 6.54 | 2.95 |
| tₘₐₓ (h) | 1.33 | 0.52 | 0.58 | 0.20 |
| AUC₍₀₋ₜ₎ | 55.86 | 15.56 | 41.45 | 14.69 |
| (ng · h/mL) | | | | |
| AUC_{(0-∞)} | 61.83 | 18.04 | 47.44 | 17.47 |
| (ng · h/mL) | | | | |
| t_{1/2} (h) | 14.36 | 1.19 | 13.50 | 1.32 |
| % F | 112.78 | N/A | 83.69 | N/A |

N/A = not applicable

TABLE C6
Summary Pharmacokinetic Parameters of Halofuginone hydrobromide
in the Plasma of Beagle Dogs Following a 0.03 mg/kg Subcutaneous
or 0.05 mg/kg Intravenous Administration
Group

| | | |
|---|---|---|
| | Week 1 | Week 2 |
| Parameter | Subcutaneo | Intraveno |

| (units) | Mean | SD | Mean | SD |
|---|---|---|---|---|
| Cₘₐₓ | 3.66 | 0.90 | 6.95 | 1.01 |
| tₘₐₓ (h) | 0.50 | 0.00 | 0.08 | 0.00 |
| AUC₍₀₋ₜ₎ | 16.22 | 7.02 | 16.51 | 7.01 |
| (ng · h/mL) | | | | |
| AUC_{(0-∞)} | 19.57 | 7.06 | 24.49 | 6.81 |
| (ng · h/mL) | | | | |
| t_{1/2} (h) | 12.63 | 4.91 | 10.35 | 1.47 |
| % F | 163.79 | N/A | N/A | N/A |

N/A = not applicable

TABLE C7
Individual Pharmacokinetic Parameters of Halofuginone hydrobromide
in the Plasma of Beagle Dogs Following a Single 0.15 mg/kg
Oral Administration as Enteric-Coated Tablets

| | Dog | | | | | | | |
|---|---|---|---|---|---|---|---|---|
| Parameter | 1 | 2 | 3 | 4 | 5 | 6 | Mean | SD |
| Cₘₐₓ | 2.56 | 4.91 | 8.28 | 5.42 | 8.34 | 6.31 | 5.97 | 2.20 |
| tₘₐₓ (h) | 1.00 | 1.00 | 2.00 | 1.00 | 1.00 | 2.00 | 1.33 | 0.52 |
| AUC₍₀₋ₜ₎ | 49.07 | 55.56 | 80.61 | 33.28 | 54.47 | 62.16 | 55.86 | 15.56 |
| (ng · h/mL) | | | | | | | | |
| AUC_{(0-∞)} | 56.23 | 60.99 | 91.22 | 35.72 | 58.74 | 68.10 | 61.83 | 18.04 |
| (ng · h/mL) | | | | | | | | |
| t_{1/2} (h) | 13.79 | 13.95 | 16.72 | 14.05 | 13.44 | 14.21 | 14.36 | 1.19 |

TABLE C8
Individual Pharmacokinetic Parameters of Halofuginone hydrobromide
in the Plasma of Beagle Dogs Following a Single 0.15 mg/kg
Oral Administration as a Clear Solution

| | Dog | | | | | | | |
|---|---|---|---|---|---|---|---|---|
| Parameter | 1 | 2 | 3 | 4 | 5 | 6 | Mean | SD |
| Cₘₐₓ | 11.57 | 3.83 | 3.78 | 6.05 | 8.15 | 5.87 | 6.54 | 2.95 |
| tₘₐₓ (h) | 0.50 | 1.00 | 0.50 | 0.50 | 0.50 | 0.50 | 0.58 | 0.20 |
| AUC₍₀₋ₜ₎ | 56.12 | 45.05 | 22.99 | 23.10 | 49.92 | 51.52 | 41.45 | 14.69 |
| (ng · h/mL) | | | | | | | | |
| AUC_{(0-∞)} | 71.29 | 50.70 | 25.02 | 28.86 | 53.32 | 55.45 | 47.44 | 17.47 |
| (ng · h/mL) | | | | | | | | |
| t_{1/2} (h) | 12.82 | 15.66 | 14.06 | 11.74 | 13.11 | 13.63 | 13.50 | 1.32 |

TABLE C9
Individual Pharmacokinetic Parameters of Halofuginone hydrobromide
in the Plasma of Beagle Dogs Following a Single 0.03 mg/kg
Subcutaneous Administration as a Clear Solution

| | Dog | | | | | | | |
|---|---|---|---|---|---|---|---|---|
| Parameter | 7 | 8 | 9 | 10 | 11 | 12 | Mean | SD |
| Cₘₐₓ | 2.82 | 2.95 | 2.98 | 5.07 | 4.29 | 3.82 | 3.66 | 0.90 |
| tₘₐₓ (h) | 0.50 | 0.50 | 0.50 | 0.50 | 0.50 | 0.50 | 0.50 | 0.00 |
| AUC₍₀₋ₜ₎ | 11.77 | 8.75 | 11.77 | 26.90 | 22.29 | 15.87 | 16.22 | 7.02 |
| (ng · h/mL) | | | | | | | | |
| AUC(0-∞) | 12.95 | 13.73 | 16.87 | 30.88 | 25.26 | 17.71 | 19.57 | 7.06 |
| (ng · h/mL) | | | | | | | | |
| t_{1/2} (h) | 7.44 | 9.08 | 16.84 | 17.27 | 17.16 | 8.00 | 12.63 | 4.91 |

TABLE 10
Individual Pharmacokinetic Parameters of Halofuginone hydrobromide
in the Plasma of Beagle Dogs Following a Single 0.05 mg/kg
Intravenous Administration as a Clear Solution

| | Dog | | | | | | | |
|---|---|---|---|---|---|---|---|---|
| Parameter | 7 | 8 | 9 | 10 | 11 | 12 | Mean | SD |
| Cₘₐₓ | 7.81 | 6.64 | 5.12 | 7.42 | 7.80 | 6.88 | 6.95 | 1.01 |
| tₘₐₓ (h) | 0.08 | 0.08 | 0.08 | 0.08 | 0.08 | 0.08 | 0.08 | 0.00 |
| AUC₍₀₋ₜ₎ | 13.50 | 12.22 | 11.70 | 30.09 | 17.98 | 13.56 | 16.51 | 7.01 |
| (ng · h/mL) | | | | | | | | |
| AUC_{(0-∞)} | 22.56 | 17.76 | 20.91 | 36.32 | 28.57 | 20.80 | 24.49 | 6.81 |
| (ng · h/mL) | | | | | | | | |
| t_{1/2} (h) | 12.32 | 8.93 | 12.04 | 10.03 | 9.65 | 9.12 | 10.35 | 1.47 |

Statistical analysis of PK parameters for dogs treated with both enteric and oral solutions were calculated using Excel® spreadsheets. Summary statistics were derived for the following PK parameters for each dog receiving both the dog EC tablets and the oral drug as a clear solution in Group 1:
Cmax (ng/mL): Maximum Observed Concentration
tmax (h): Time Point at Cₘₐₓ
AUC(0-t) (ng·h/mL): AUC to the last non-zero concentration (t is the corresponding time)
AUC(0-∞) (ng·h/mL): AUC_{(0-∞)}=AUC₍₀₋ₜ₎+AUC_{(t-∞)}
t_{1/2} (h): Half-life; time taken for drug plasma concentration to fall by one-half
% F: Bioavailability; [AUC₍₀₋ₜ₎ X*Dose IV (mg/kg)]/[AUC₍₀₋ₜ₎ IV*Dose X (mg/kg)]
   Paired differences for each dog were determined by subtracting the summary PK value as follows:

Summary PK (Dog Enteric Coated Tablets)−Summary PK (Drug Oral as Clear Solution)

The descriptive statistics for each of the paired differences was determined and a one sample t-test was performed testing the null hypothesis that the mean of the difference was equal to zero versus the alternative that it is not equal to zero.

Table C15 presents the results of the descriptive statistics and the p-values of the one sample t-test. In addition a Wilcoxon signed rank test was also performed but is not presented in the table. The results from Table C15 indicate that the tₘₐₓ (h) for the paired difference t-test was statistically significant (p=0.03). The 95% Confidence interval is (0.107, 1.393). The paired differences for the other PK parameters were not statistically significant.

TABLE C15
Summary of Descriptive Statistics: Change of PK Parameter for
Dog Enteric- Coated Tablets - Drug Oral as Clear Solution

| PK | | | | | | | p- |
|---|---|---|---|---|---|---|---|
| Parameter | N | Mean | Median | SD | Min | Max | value |
| Cₘₐₓ | 6.000 | −0.572 | 0.315 | 4.500 | −9.010 | 4.500 | 0.77 |
| (ng/mL) | | | | | | | |
| tₘₐₓ | 6.000 | 0.750 | 0.500 | 0.612 | 0.000 | 1.500 | 0.03 |
| AUC₍₀₋ₜ₎ | 6.000 | 14.408 | 10.345 | 22.236 | −7.050 | 57.620 | 0.17 |
| AUC_{(0-∞)} | 6.000 | 14.393 | 8.575 | 27.234 | −15.060 | 66.200 | 0.25 |
| t_{1/2} | 6.000 | 0.857 | 0.775 | 1.570 | −1.710 | 2.660 | 0.24 |

The p-value for the Wilcoxon signed rank test with continuity correction testing the paired difference of the time to tₘₐₓ (h) was p=0.053 which is still borderline statistically significant.

### Statistical Summary

Overall, there is a clear trend showing an extended time to maximum concentration (tₘₐₓ) with the tablets and higher exposure with the EC vs. a solution at the same relative dose level.
Based on a paired t-test, the data on the Group 1 dogs extended the time to maximum concentration (tₘₐₓ) by 0.75 h on average (p=0.03) when treated with the EC tablets compared with a solution at the same relative dose level.
The average Cₘₐₓ (ng/mL) of the paired differences was slightly lower in the EC tablets compared with the oral formulation as a clear solution. However, the difference was not statistically significant.
Paired differences for other PK parameters suggest that there was a slight trend for the AUC summary statistics to be higher for the EC tablets compared with the oral formulation as a clear solution, but this was not statistically significant.

### Results

Halofuginone hydrobromide was well tolerated when dosed as a SC or IV solution. It was tolerated with some GI effects noted when dosed orally as EC tablets and as a solution, with substantially fewer GI effects observed with administration of the EC tablets vs. the oral solution. (6 episodes of emesis for the EC tablets vs. 18 for the capsules).

The absolute bioavailability of Halofuginone hydrobromide was increased when it was dosed as EC tablets and as a subcutaneous solution versus being dosed as an oral solution. This may be a result of the frequent emesis observed after dosing of the oral solution.

Overall there is a clear trend showing an extended time to tₘₐₓ with the tablets and a higher exposure with the EC tablets vs. the solution. Other PK parameters had some trends but did not achieve statistically significant conclusions.

The EC tablets again showed improved tolerability over the oral dosing solution as was observed in a previous PK study. Where 5 of 6 animals dosed with the solution had emesis, only 2 showed this effect when given a similar dose via EC tablets (in spite of the increase in bioavailability). In addition, there were a total of 18 separate emetic events recorded in the solution arm but only 6 in the arm receiving EC tablets.

### CONCLUSION

Systemic exposure to Halofuginone hydrobromide was demonstrated using EC capsules (˜0.15 mg/kg), a PO solution (0.15 mg/kg), a SC solution (0.03 mg/kg), and an IV solution (0.05 mg/kg). Halofuginone hydrobromide was better tolerated as a SC or IV solution when compared to both PO doses. For the orally-administered formulations, the EC tablets were also better tolerated than the same dose given via solution. The tₘₐₓ was significantly extended with EC capsules as compared to the PO solution. However, a downward trend was noticed for Cₘₐₓ. The area under the time versus concentration curve (AUC) was slightly higher using EC capsules as compared to the PO solution. The half life (t_{1/2}) was similar between the two PO formulations and between the SC and IV solutions. The bioavailabilty was higher in the EC tablets and the SC dose as compared to the PO solution.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1-11. (canceled)

12. A method of treating a muscular dystrophy disorder in a human patient in need thereof, the method comprising administering to the patient an enteric-coated oral dosage form comprising 0.05 to 2 mg of halofuginone, or a pharmaceutically acceptable salt thereof, to thereby treat the disorder.

13-14. (canceled)

15. The method of claim 12, wherein the pharmaceutically acceptable salt of halofuginone is halofuginone hydrobromide.

16. The method of claim 12 wherein the dosage form is administered one or two times daily.

17. The method of claim 12 wherein the dosage form is administered one time daily.

18. The method of claim 12 wherein the dosage form is administered two times daily.

19-25. (canceled)

26. The method of claim 12, wherein the muscular dystrophy is Duchenne MD.

27-31. (canceled)

32. The method of claim 12, wherein the enteric coating comprises a wax, shellac, polymer, or plant fiber.

33. The method of claim 32, wherein the enteric coating comprises a polymer.

34. The method of claim 33, wherein the polymer is a co-polymer.

35. The method of claim 34, wherein the co-polymer comprises methacrylic acid or ethylacrylic acid.

36. The method of claim 34, wherein the co-polymer comprises poly(methacrylic acid-co-ethyl acrylate).

37-44. (canceled)
